(19) United States
(12) Patent Application Publication
(10) Pub. No.: US 2024/0083946 A1
(43) Pub. Date: Mar. 14, 2024

(54) SELF-ASSEMBLING PEPTIDES, NANOFIBERS, AND METHODS OF USE

(71) Applicant: The Board of Regents of the University of Oklahoma, Norman, OK (US)

(72) Inventor: Handan Acar, Norman, OK (US)

(21) Appl. No.: 18/280,351

(22) PCT Filed: Mar. 9, 2022
(86) PCT No.: PCT/US2022/019621

§ 371 Date: Sep. 5, 2023

Related U.S. Application Data
(60) Provisional application No. 63/159,050, filed on Mar. 10, 2021.

(51) Int. Cl. C07K 7/08; A61K 39/00; A61K 39/39

(52) U.S. Cl. CPC C07K 7/08; A61K 39/0011; A61K 39/39; A61K 2039/55516

(57) ABSTRACT

Compositions of anionic and cationic peptides which co-assemble under suitable conditions to form peptide nanostructures, methods of assembling the peptide nanostructures, and methods of use of the peptide nanostructures in hydrogels and as vaccines and vaccine adjuvants. The peptide nanostructures demonstrate stability once self-assembled and are biocompatible and have therapeutic functionality, particularly when equipped with additional functional features such as ligands, fluorophores, antigens, drugs, or other bioactive compounds.

### CROSS-REFERENCE TO RELATED APPLICATIONS

The present application claims the benefit of U.S. Provisional Patent Application Ser. No. 63/159,050 filed Mar. 10, 2021, the disclosure of which is hereby expressly incorporated by reference in its entirety.

### BACKGROUND

Synthetic peptides with the ability to self-assemble into supramolecular nanofibers are known. Such nanofibers have been used in laboratory and clinical applications, including cell culture, drug delivery, accelerated cartilage and bone growth, and regeneration of tissues, and as a matrix, scaffold, or tether that can be associated with one or more detectable agents, therapeutic agents, biologically active agents, cells, and/or cellular components. However, improved control of self-assembling peptides relating to combination compositions, particularly comprising payloads and cargo molecules, such as therapeutic agents, are necessary.

### BRIEF DESCRIPTION OF THE DRAWINGS

Several embodiments of the present disclosure are hereby illustrated in the appended drawings. It is to be noted, however, that the appended drawings only illustrate several typical embodiments and are therefore not intended to be considered limiting of the scope of the disclosure. The figures are not necessarily to scale and certain features and certain views of the figures may be shown as exaggerated in scale or in schematic in the interest of clarity and conciseness. The patent or application file contains at least one drawing executed in color. Copies of this patent or patent application publication with color drawing(s) will be provided by the Office upon request and payment of the necessary fee.

FIG. 1 shows a schematic diagram of a peptide nanostructure (nanofiber) that can be formed from the self-assembly of anionic and cationic peptides of the present disclosure. The nanostructure comprises a plurality of peptide segments configured in a “stacked” (“side-by-side lengthwise”) orientation to form an elongated nanofiber. Segments with a (+) denote a peptide segment having an overall positive charge (a cationic peptide segment). Segments with a (−) denote a peptide segment having an overall negative charge (an anionic peptide segment). Cationic peptides alternate with anionic peptides. The n corresponds to any number of additional cationic or anionic peptides.

FIG. 2 shows the schematic nanostructure of FIG. 1 having cargo molecules “C” linked by a linker “L” to termini of several cationic peptides of the nanostructure.

FIG. 3 shows the schematic nanostructure of FIG. 1 having cargo molecules “C” linked by a linker “L” to termini of several anionic peptides of the nanostructure.

FIG. 4 shows the schematic nanostructure of FIG. 1 having cargo molecules “C” linked by a linker “L” to termini of several anionic peptides and cationic peptides of the nanostructure.

FIG. 5A shows chemical structures of three anionic/cationic peptides sets used in experiments here. E and K residues at both termini provide electrostatic interactions, an FF pair at the core contributed self-assembly with pi-stacking, and the position X was either AA, WW, or II (see Table 5 for amino acid sequences of each peptide). Four other peptide sets which used FF, VV, LL, and GG in the X position were also tested. The seven sets of peptides provided tunable hydrophobic interactions.

FIG. 5B shows results of combining the AA, II, and WW self-assembling peptide sets, respectively, of FIG. 5A. Upon mixing, anionic/cationic peptides comprising II and WW, respectively, self-assembled into gels comprising nanofibers.

FIG. 5C shows micrographs of nanofibers formed from the co-assembling anionic/cationic peptides of FIG. 5A.

FIG. 6A shows measurements of relative ATP in OVCAR-8 cells exposed to different doses of [AA], [WW], and [II] co-assembling peptide sets after 6 hours. Dead cells reduce ATP.

FIG. 6B shows images of OVCAR-8 cells exposed to [AA], [WW], and [II] co-assembling peptide sets and individual peptide types after 6 hours. Image analysis indicated green cells were living and red cells were dead (color not shown). SEQ ID NOS for each peptide segment are shown in Table 5.

FIG. 6C shows time-dependency of toxicity of OVCAR cells to the [II] co-assembling peptide set (0.5 mM).

FIG. 6D shows pyroptotic morphology of the treated OVCAR cells of FIG. 6C after 6 hours, and propidium iodide staining of the cells at 6 h.

FIG. 6E shows western blot analysis of pro-caspase-3 and cleaved caspase-3 at 6 hours for the treated cells of FIG. 6C. Beta-actin was used as housekeeping loading control.

FIG. 7A shows self-assembled nanofibers which have been treated with citrate-coated gold nanoparticles (AuNPs). Binding of the AuNPs to the nanofibers is minimal.

FIG. 7B shows self-assembled nanofibers which have been conjugated to ovalbumin protein (OVA). When the OVA-conjugated nanofibers are treated with citrate-coated AuNPs, the AuNPs readily bind to the nanofibers.

FIG. 8A shows results of an in vivo analysis of OVA-conjugated nanofibers (“conj. OVA pep”) in terms of anti-OVA IgG1 response. Antibody production against OVA in mice vaccinated with OVA-[II] nanofiber mixture.

FIG. 8B shows that after a second vaccination, a higher amount of antibody production was observed, not only with the OVA-[II] nanofiber mixture, but also in the OVA-conjugated nanofiber treatment group.

FIG. 9 shows the scheme for synthesizing an OVA-linker-self-assembling [II] peptide conjugate.

### DETAILED DESCRIPTION

The present disclosure is directed to co-assembling cationic and anionic peptides, compositions of such cationic and anionic peptides, organized nanostructures (nanofibers) assembled from such cationic and anionic peptides, and methods of use of the cationic and anionic peptides and of the nanostructures assembled therefrom. The disclosed self-assembling peptides when combined in a mixture are able to spontaneously organize into molecules having a precise supramolecular architecture, such as beta-sheet nanofibers, when subjected to suitable conditions. These nanofibers demonstrate stability once self-assembled and are biocompatible. They also have therapeutic functionality, particularly when equipped with additional functional features such as ligands, fluorophores, antigens, drugs, or other bioactive compounds. Functional capabilities can be installed directly into the nanostructures via covalent fusion of a functional molecule to the self-assembling peptides. It can also be encapsulated by simply mixing them.

Before further describing various embodiments of the compositions and methods of the present disclosure in more detail by way of exemplary description, examples, and results, it is to be understood that the embodiments of the present disclosure are not limited in application to the details of methods and compositions as set forth in the following description. The embodiments of the compositions and methods of the present disclosure are capable of being practiced or carried out in various ways not explicitly described herein. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. While the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts as described herein. All such similar substitutes and modifications apparent to those having ordinary skill in the art are deemed to be within the spirit and scope of the inventive concepts as disclosed herein.

All patents, published patent applications, and non-patent publications referenced or mentioned in any portion of the present specification are indicative of the level of skill of those skilled in the art to which the present disclosure pertains, and are hereby expressly incorporated by reference in their entirety to the same extent as if the contents of each individual patent or publication was specifically and individually incorporated herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” are used to indicate that a value includes the inherent variation of error for the composition, the method used to administer the composition, or the variation that exists among the objects, or study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, percentage, temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, as well as 1.1, 1.2, 1.3, 1.4, 1.5, etc., 2.1, 2.2, 2.3, 2.4, 2.5, etc., and so forth, where the range is not limited solely to integers. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability of an active agent to modify the physiological system of an organism without reference to how the active agent has its physiological effects.

As used herein, “pure,” “substantially pure,” or “isolated” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure. Where used herein the term “high specificity” refers to a specificity of at least 90%, or at least 91%, or at least 92%, or at least 93%, or at least 94%, or at least 95%, or at least 96%, or at least 97%, or at least 98%, or at least 99%. Where used herein the term “high sensitivity” refers to a sensitivity of at least 90%, or at least 91%, or at least 92%, or at least 93%, or at least 94%, or at least 95%, or at least 96%, or at least 97%, or at least 98%, or at least 99%.

The terms “subject” and “patient” are used interchangeably herein and will be understood to refer an organism to which the compositions of the present disclosure are applied and used, such as a vertebrate or more particularly to a warm-blooded animal, such as a mammal. Non-limiting examples of animals within the scope and meaning of this term include dogs, cats, rats, mice, guinea pigs, chinchillas, horses, goats, cattle, sheep, llamas, zoo animals, Old and New World monkeys, non-human primates, and humans.

“Treatment” refers to therapeutic treatments, such as for healing or restoration of damaged tissues. The term “treating” refers to administering the composition to a patient such therapeutic purposes, and may result in an amelioration of the condition or disease.

The terms “therapeutic composition” and “pharmaceutical composition” refer to an active agent composition, such as the hydrogel compositions described herein, that may be administered to a subject by any method known in the art or otherwise contemplated herein, wherein administration of the composition brings about a therapeutic effect as described elsewhere herein. In addition, certain compositions of the present disclosure may be designed to provide targeted, delayed, controlled, extended, and/or sustained release using formulation techniques which are well known in the art.

The term “effective amount” refers to an amount of an active agent which is sufficient to exhibit a detectable biochemical and/or therapeutic effect, for example without excessive adverse side effects (such as toxicity, irritation and allergic response) commensurate with a reasonable benefit/risk ratio when used in the manner of the present disclosure. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by a person of ordinary skill in the art using routine experimentation based on the information provided herein.

The term “ameliorate” means a detectable or measurable improvement in a subject's condition or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one of, most of, or all of the adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

As used herein, the terms “attached,” “attachment,” “connected,” and the like can refer to the formation of a covalent or non-covalent association (e.g., a bond) between two or more molecules or conjugation of two or more molecules. As used herein, “attached,” “attachment” and the like can refer to direct association of two or more molecules together with no intermediate molecules between those that are attached together or to the indirect attachment of two or more molecules together that is mediated via one or more linkers. Where the association is non-covalent, this can encompass charge interactions, affinity interactions, metal coordination, physical adsorption, host-guest interactions, hydrophobic interactions, pi-pi stacking interactions, hydrogen bonding interactions, van der Waals interactions, magnetic interactions, electrostatic interactions, dipole-dipole interactions, and/or combinations thereof. Where the association is covalent, this can encompass bonds where a pair of electrons is shared between one or more atoms in each molecule involved. As used herein, the term “coupled” and/or “conjugated” can refer to the direct or indirect (e.g., via a linker) attachment of two or more molecules and/or compounds.

Specific amino acids (i.e., the “common natural amino acids”) may be referred to herein by the following designations: alanine: ala or A; arginine: arg or R; asparagine: asn or N; aspartic acid: asp or D; cysteine: cys or C; glutamic acid: glu or E; glutamine: gln or Q; glycine: gly or G; histidine: his or H; isoleucine: ile or I; leucine: leu or L; lysine: lys or K; methionine: met or M; phenylalanine: phe or F; proline: pro or P; serine: ser or S; threonine: thr or T; tryptophan: trp or W; tyrosine: tyr or Y; and valine: val or V.

Cationic amino acids used herein can have positively charged side (or “R” groups) groups and include, but are not limited to, lysine, arginine, and histidine. Anionic amino acids used herein can have negatively charged side groups and can include, but are not limited to, aspartate and glutamate. Polar amino acids can have polar, uncharged side groups, and can include, but are not limited to, serine, threonine, cysteine, proline, asparagine, and glutamine. Hydrophobic amino acids can have nonpolar, aliphatic or aromatic side groups and can include, but are not limited to, glycine, alanine, valine, leucine, methionine, isoleucine, phenylalanine, tyrosine, and tryptophan.

Amino acids which may be used to make the peptides of the present disclosure include the natural amino acids, such as alanine, cysteine, aspartic acid, glutamic acid, phenylalanine, glycine, histidine, isoleucine, lysine, leucine, methionine, asparagine, proline, glutamine, arginine, serine, threonine, valine, tryptophan, and tyrosine, and D-amino acids forms thereof, and uncommon or nonnatural amino acids (including L- or D-amino acid forms). “Nonnatural amino acid” as used herein refers to any amino acid which is not a natural amino acid. This includes, for example, amino acids that comprise alpha-, beta-, gamma-, D-, and L-amino acyl residues. More generally, the nonnatural amino acid comprises a residue wherein the side chain is other than the amino acid side chains occurring in nature.

Exemplary nonnatural amino acids, include, but are not limited to, allothreonine, alpha-asparagine, alpha-methylleucine, alpha-methylproline, alpha-methylphenylalanine, 2-aminobutanoic acid, 2-aminobutyric acid, 4-aminobutyric acid, 2-aminocapric acid (2-aminodecanoic acid), 6-aminocaproic acid, alpha-glutamine, 2-aminoheptanoic acid, 6-aminohexanoic acid, alpha-aminoisobutyric acid (2-aminoalanine), 3-aminoisobutyric acid, beta-alanine, allo-hydroxylysine, allo-isoleucine, 4-amino-7-methylheptanoic acid, 4-amino-5-phenylpentanoic acid, 2-aminopimelic acid, 2-aminosuberic acid, 2-carboxyazetidine, beta-aspartic acid, beta-ureidoalanine, biphenylalanine, 3,6-diaminohexanoic acid, butanoic acid, citrulline, cyclobutyl alanine, cyclohexylglycine, cyclohexylalanine, beta-cyclohexylalanine, cyclopentyl alanine, beta-cyclopentylalanine, N5-aminocarbonylornithine, cyclopropyl alanine, 3-sulfoalanine, 2,4-diaminobutanoic acid, diaminopropionic acid, 2,4-diaminobutyric acid, diphenyl alanine, NN-dimethylglycine, diaminopimelic acid, 2,3-diaminopropanoic acid, S-ethylthiocysteine, N-ethylasparagine, N-ethylglycine, gamma-amino-beta-hydroxybenzenepentanoic acid, gamma-glutamic acid, gamma-carboxyglutamic acid, hydroxyacetic acid, pyroglutamic acid, homoarginine, homocitrulline, homocysteic acid, homocysteine, homohistidine, 2-hydroxyisovaleric acid, homoleucine, homoproline (pipecolic acid), homoserine, homophenylalanine, 2-hydroxypentanoic acid, hydroxyproline, 4-hydroxyproline, 2-carboxyoctahydroindole, 3-carboxyisoquinoline, isovaline, 2-hydroxypropanoic acid (lactic acid), mercaptoacetic acid, mercaptobutanoic acid, sarcosine, 4-methyl-3-hydroxyproline, mercaptopropanoic acid, norleucine, nipecotic acid, nortyrosine, norvaline, omega-amino acid, ornithine, penicillamine (3-mercaptovaline), tert-butylglycine, tert-butylalanine, phenylglycine, 2-phenylglycine, 2-carboxypiperidine, sarcosine (N-methylglycine), 2-amino-3-(4-sulfophenyl)propionic acid, 1-amino-1-carboxycyclopentane, 3-thienylalanine, epsilon-N-trimethyllysine, 3-thiazolylalanine, thiocitrulline, thiazolidine 4-carboxylic acid, alpha-amino-2,4-dioxopyrimidinepropanoic acid, 2-naphthylalanine, 4-aza-phenylalanine, p-fluorophenylalanine, o-fluorophenylalanine, homophenylalanine, bromophenylalanine, 6-bronotryptophan, 5-bromotryptophan. 7-azatryptophan, and 5-hydroxytryptophan, in-luorophenylalanine, 6-chlorotryptophan, and benzothienylalanine.

The term “hydrogel”, as used herein, refers, in non-limiting embodiments, to a water-soluble network of functionalized or non-functionalized nanofibers made from the anionic/cationic peptide compositions disclosed herein. The network of nanofibers may be cross-linked via covalent interactions or may be a network held together via non-covalent, hydrostatic interactions.

As used herein, “cDNA” can refer to a synthetic DNA sequence that is complementary to an RNA transcript in a cell. Typically, cDNA is made in vitro by an enzyme called reverse-transcriptase using RNA transcripts as templates.

As used herein, “chemotherapeutic agent” or “chemotherapeutic” can refer to a therapeutic agent utilized to prevent or treat a cancer.

As used herein, the term “linker” can refer to molecule which can serve as a linkage between two other molecules of structures. For example, a linker may be any amino acid or peptide that can be included between a positive or negative peptide segment and a cargo molecule such as a peptide or protein. Linker peptides can range in length from about 1 to about 60 amino acids. The linker can be composed of any of the 20 naturally occurring amino acids or non-natural amino acids such as D-amino acids and can be present in any arrangement that does not otherwise perturb the peptide segment assembly or cargo molecule activity.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions. When used in reference to the peptides of the present disclosure, molecular weight refers to a mass of 1 mol of peptide molecules.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same term can be applied to the arrangement of coding sequences and/or transcription control elements (e.g., promoters, enhancers, and termination elements), and/or selectable markers in an expression vector. “Operatively linked” can also refer to an indirect attachment (i.e., not a direct fusion) of two or more polynucleotide sequences or polypeptides to each other via a linking molecule (also referred to herein as a linker).

In certain embodiments, the nanofibers made by the anionic/cationic peptide compositions disclosed herein may be functionalized with cell adhesion peptides such as integrin-targeting peptides (“RGD” peptides). An RGD peptide refers to a peptide having the three amino acid residue motif “arginine-glycine-aspartic acid,” and having cell adhesion properties. Other, RGD-peptidomimetic constructs and non-RGD cell adhesion peptides may be used instead. Examples of RGD peptides and RGD-peptidomimetics which may be used herein include, but are not limited to, those shown in U.S. Pat. Nos. 9,115,170; 10,689,415; 11,096,997; and 11,150,251.

Peptides may be used herein as linkers for connecting cargo molecules to the anionic and cationic peptides disclosed herein. In one embodiment, the linker has the sequence GCGYG. However, any other suitable linker sequence may be used as long as it enables the cargo molecule to retain its desired activity. Non-limiting examples of peptide linker sequences which may be used herein include, but are not limited to, those shown in U.S. Pat. Nos. 9,409,950; 9,827,272; and 9,937,256. In certain embodiments the linker peptide comprises a cysteine residue. The linker peptide may be constructed to include from 1 to 25, or more, amino acid residues selected from the 20 “natural amino acids” (or any other amino acid that enables the linker to function in accordance with the present embodiments).

As used herein, the terms “cargo molecule,” “payload,” and “drug payload,” can refer to any molecule, drug, peptide, polypeptide, or compound that can be coupled to the N-terminus and/or the C-terminus of a positive or negative peptide segment as disclosed herein. The cargo molecule can be coupled to the positive or negative peptide segment using standard chemistry or molecular biology.

In one example, in which the cargo molecule is a peptide or polypeptide, the cargo molecule can be coupled to the positive or negative peptide segment using a recombinant DNA technology technique. For example, a fusion peptide segment containing a cargo polypeptide, can be produced from a recombinant DNA construct containing DNA encoding the negative or positive peptide segment operatively coupled with DNA encoding the cargo polypeptide and any optional peptide linker. The DNA encoding the negative or positive peptide segment can be operatively coupled to the cargo polypeptide and any optional peptide linker such that the cargo polypeptide is translated in-frame with the negative or positive peptide segment. The cargo polypeptide can be a reporter protein (e.g. a fluorescent protein), a pharmaceutically relevant protein (a protein that can be effective to prevent or treat a disease or symptom thereof in a subject), a cell- or tissue-targeting protein, an antibody or fragment thereof, an antigen, an enzyme, a growth factor, a cytokine, a chemokine, an extracellular matrix protein or fragment thereof, a transmembrane receptor or fragment thereof, a toxin or a fragment thereof, and a transcription factor or fragment thereof.

As noted, the cargo molecule may be a peptide, oligopeptide, or polypeptide coupled to the anionic and/or cationic peptide segments. The cargo polypeptide can be coupled directly (e.g. no amino acids existing between the N terminus of the peptide segment and the C-terminus of the cargo polypeptide) to the peptide segment, or indirectly, e.g. via an optional linker. In certain embodiments the linker can be any amino acid sequence ranging from 1 to 60 amino acids. The linker can be composed of any of the amino acids described elsewhere herein that does not perturb the assembly behavior of the peptide segment and/or the bioactivity of the cargo molecule.

The anionic and cationic peptide segments can be produced from nucleic acids (e.g., DNA or RNA) that encode the anionic and cationic peptide segments. Based on the amino acid sequences provided herein, one of ordinary skill in the art will know techniques and methods that will enable them to generate suitable coding nucleic acid sequences for the peptide segments. In some embodiments, the nucleic acids that encode the positive and negative peptide segments can be codon optimized for expression in a particular cell type, such as E. coli. The nucleic acids encoding the peptide segment(s) can be included in a suitable expression vector, as understood by those of ordinary skill in the art. In some embodiments, the expression vector can also express genes that can result in more efficient and/or accurate protein folding and other post-translation modifications. Such expression vectors will be appreciated by those of ordinary skill in the art. The expression vectors can be introduced into a suitable cell and the polypeptides can be produced by expression in the cells and harvested using techniques generally known in the art.

In one non-limiting embodiment, a fusion peptide segment containing the cargo polypeptide, can be produced from a recombinant DNA construct containing DNA encoding the peptide segments operatively coupled with DNA encoding the cargo polypeptide and any optional linker. The cargo polypeptide can be a reporter protein (e.g. a fluorescent protein), a pharmaceutically relevant protein (e.g., a protein or peptide that can be effective to prevent or treat a disease or symptom thereof in a subject), a cell- or tissue-targeting protein, an antibody or fragment thereof, enzyme, growth factor, cytokine, chemokine, extracellular matrix protein or fragment thereof, structural protein or fragment thereof, a transmembrane protein or fragment thereof, a transcription factor or fragment thereof, and/or an antigen.

Generally, the peptide segments do not self-assemble into nanofibers until both the anionic and cationic peptide segments are present together under stimulating conditions. In some embodiments, the stimulating conditions can be incubation and/or placement in a solution (e.g., an aqueous solution) at about a neutral or near physiological pH. In some embodiments, the pH of the solution can range from about 6.5 to about 8.5, or from about 6.5 to about 7.5. Once stimulated, the ionizable amino acids are ionized and the peptide segments self-assemble into beta sheets with alternating positive and negative segments. The peptide segments (with and/or without a cargo molecule) can self-assemble into structures, such as nanofibrillar hydrogels, nanofibers, microparticles, or nanoparticles, depending, for example, on the concentration of the peptide segments.

In certain embodiments, the nanofibers assembled from the peptide compositions can be incorporated into other biomaterials and compositions including, but not limited to, hydrogels, synthetic polymer matrices or network, natural polymer matrices or networks, composite networks of natural and synthetic polymers, polymer nanoparticles, and/or polymer microparticles. The term “nanofiber” where used herein refers to a nanostructure comprising a plurality of cationic and anionic peptides organized into a “stacked” structure, wherein the cationic peptides alternate with anionic peptides in a sandwich (side-by-side lengthwise) configuration, such that the axis of the resulting nanofiber is substantially perpendicular to the axes of the assembled peptides in the nanofiber.

The anionic and cationic peptides and nanofibers of the present disclosure may be conjugated to or coalesced with one or more cargo molecules such as therapeutic agents and diagnostic agents, including but not limited to antibiotics, antibodies or antigen-binding fragments of antibodies, anti-cancer agents, small molecules, peptides, RNAs, DNAs, aptamers, radioisotopes, and imaging agents. Exemplary therapeutic agents include, but are not limited to: anti-proliferative/antimitotic agents including natural products such as vinca alkaloids (i.e., vinblastine, vincristine, and vinorelbine), paclitaxel, epidipodophyllotoxins (i.e., etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) II_{b}/HI a inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e., estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives e.g., aspirin; para-aminophenol derivatives, e.g., acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), everolimus, azathioprine, mycophenolate mofetil); angiogenic agents: vascular endothelial growth factor (VEGF), fibroblast growth factor (FGF); angiotensin receptor blockers; nitric oxide donors; antisense oligonucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors; beta₂ agonists (e.g., salbutamol, terbutaline, clenbuterol, salmeterol, formoterol); steroids such glycocorticosteroids, preferably anti-inflammatory drugs (e.g., Ciclesonide, Mometasone, Flunisolide, Triamcinolone, Beclomethasone, Budesonide, Fluticasone); anticholinergic drugs (e.g., ipratropium, tiotropium, oxitropium); leukotriene antagonists (e.g., zafirlukast, montelukast, pranlukast); xantines (e.g., aminophylline, theobromine, theophylline); Mast cell stabilizers (e.g., cromoglicate, nedocromil); inhibitors of leukotriene synthesis (e.g., azelastina, oxatomide ketotifen); mucolytics (e.g., N-acetylcysteine, carbocysteine); antibiotics, (e.g., Aminoglycosides such as, amikacin, gentamicin, kanamycin, neomycin, netilmicin streptomycin, tobramycin; Carbacephem such as loracarbef, Carbapenems such as ertapenem, imipenem/cilastatin meropenem; first generation cephalosporins such as cefadroxil, cefaxolin, and cephalexin; second generation cephalosporins such as cefaclor, cefamandole, defoxitin, cefproxil, and cefuroxime; third generation cephalosporins such as cefixime, cefdinir, ceftaxidime, defotaxime, cefpodoxime, and ceftriaxone; fourth generation cephalosporins such as maxipime; Glycopeptides such as vancomycin, teicoplanin; Macrolides such as azithromycin, clarithromycin, Dirithromycin, Erythromycin, troleandomycin; Monobactam such as aztreonam; Penicillins such as Amoxicillin, Ampicillin, Azlocillin, Carbenicillin, Cloxacillin, Dicloxacillin, Flucloxacillin, Mezlocillin, Nafcillin, Penicillin, Piperacillin, Ticarcillin; Polypeptides such as bacitracin, colistin, polymyxin B; Quinolones such as Ciprofloxacin, Enoxacin, Gatifloxacin, Levofloxacin, Lomefloxacin, Moxifloxacin, Norfloxacin, Ofloxacin, Trovafloxacin; Sulfonamides such as Mafenide, Prontosil, Sulfacetamide, Sulfamethizole, Sulfanamide, Sulfasalazine, Sulfisoxazole, Trimethoprim, Trimethoprim-Sulfamethoxazole Co-trimoxazole (TMP-SMX); Tetracyclines such as Demeclocycline, Doxycycline, Minocycline, Oxytetracycline, Tetracycline; Others such as Chloramphenicol, Clindamycin, Ethambutol, Fosfomycin, Furazolidone, Isoniazid, Linezolid, Metronidazole, Nitrofurantoin, Pyrazinamide, Quinupristin/Dalfopristin, Rifampin, Spectinomycin); pain relievers in general such as analgesic and antiinflammatory drugs, including steroids (e.g., hydrocortisone, cortisone acetate, prednisone, prednisolone, methylprednisolone, dexamethasone, betamethasone, triamcinolone, beclometasone, fludrocortisone acetate, deoxycorticosterone acetate, aldosterone); and non-steroid anti-inflammatory drugs (e.g., salicylates such as aspirin, amoxiprin, benorilate, coline magnesium salicylate, diflunisal, faislamine, methyl salicylate, salicyl salicylate); Arylalkanoic acids such as diclofenac, aceclofenac, acematicin, etodolac, indometacin, ketorolac, nabumetone, sulindac tolmetin; 2-Arylpropionic acids (profens) such as ibuprofen, carprofen, fenbufen, fenoprofen, flurbiprofen, ketoprofen, loxoprofen, naproxen, tiaprofenic acid; N-arylanthranilic acids (fenamic acids) such as mefenamic acid, meclofenamic acid, tolfenamic acid; Pyrazolidine derivatives such as phenylbutazone, azapropazone, metamizole, oxyphenbutazone; Oxicams such as piroxicam, meloxicam, tenoxicam; Coxib such as celecoxib, etoricoxib, lumiracoxib, parecoxib, rofecoxib (withdrawn from market), valdecoxib (withdrawn from market); Sulphonanilides such as nimesulide; others such as licofelone, omega-3 fatty acids; cardiovascular drugs such as glycosides (e.g., strophantin, digoxin, digitoxin, proscillaridine A); respiratory drugs; antiasthma agents; bronchodilators (adrenergics: albuterol, bitolterol, epinephrine, fenoterol, formoterol, isoetharine, isoproterenol, metaproterenol, pirbuterol, procaterol, salmeterol, terbutaline); anticancer agents (e.g., cyclophosphamide, doxorubicine, vincristine, methotrexate); alkaloids (i.e., ergot alkaloids) or triptans such as sumatriptan, rizatriptan, naratriptan, zolmitriptan, eletriptan and almotriptan, than can be used against migraine; drugs (i.e., sulfonylurea) used against diabetes and related dysfunctions (e.g., metformin, chlorpropamide, glibenclamide, glicliazide, glimepiride, tolazamide, acarbose, pioglitazone, nateglinide, sitagliptin); sedative and hypnotic drugs (e.g., Barbiturates such as secobarbital, pentobarbital, amobarbital; uncategorized sedatives such as eszopiclone, ramelteon, methaqualone, ethchlorvynol, chloral hydrate, meprobamate, glutethimide, methyprylon); psychic energizers; appetite inhibitors (e.g., amphetamine); antiarthritis drugs (e.g., NSAIDs); antimalaria drugs (e.g., quinine, quinidine, mefloquine, halofantrine, primaquine, cloroquine, amodiaquine); antiepileptic drugs and anticonvulsant drugs such as Barbiturates, (e.g., Barbexaclone, Metharbital, Methylphenobarbital, Phenobarbital, Primidone), Succinimides (e.g., Ethosuximide, Mesuximide, Phensuximide), Benzodiazepines, Carboxamides (e.g., Carbamazepine, Oxcarbazepine, Rufinamide) Fatty acid derivatives (e.g., Valpromide, Valnoctamide); Carboxilyc acids (e.g, Valproic acid, Tiagabine); Gaba analogs (e.g., Gabapentin, Pregabalin, Progabide, Vigabatrin); Topiramate, Ureas (e.g., Phenacemide, Pheneturide), Carbamates (e.g., emylcamate Felbamate, Meprobamate); Pyrrolidines (e.g., Levetiracetam Nefiracetam, Seletracetam); Sulfa drugs (e.g., Acetazolamide, Ethoxzolamide, Sultiame, Zonisamide) Beclamide; Paraldehyde, Potassium bromide; antithrombotic drugs such as Vitamin K antagonists (e.g., Acenocoumarol, Dicumarol, Phenprocoumon, Phenindione, Warfarin); Platelet aggregation inhibitors (e.g., antithrombin III, Bemiparin, Deltaparin, Danaparoid, Enoxaparin, Heparin, Nadroparin, Pamaparin, Reviparin, Tinzaparin); other platelet aggregation inhibitors (e.g., Abciximab, Acetylsalicylic acid, Aloxiprin, Ditazole, Clopidogrel, Dipyridamole, Epoprostenol, Eptifibatide, Indobufen, Prasugrel, Ticlopidine, Tirofiban, Treprostinil, Trifusal); Enzymes (e.g., Alteplase, Ancrod, Anistreplase, Fibrinolysin, Streptokinase, Tenecteplase, Urokinase); Direct thrombin inhibitors (e.g., Argatroban, Bivalirudin. Lepirudin, Melagatran, Ximelagratan); other antithrombotics (e.g., Dabigatran, Defibrotide, Dermatan sulfate, Fondaparinux, Rivaroxaban); antihypertensive drugs such as Diuretics (e.g., Bumetanide, Furosemide, Torsemide, Chlortalidone, Hydrocloro thiazide, Chlorothiazide, Indapamide, metolaxone, Amiloride, Triamterene); Antiadrenergics (e.g., atenolol, metoprolol, oxprenolol, pindolol, propranolol, doxazosin, prazosin, teraxosin, labetalol); Calcium channel blockers (e.g., Amlodipine, felodipine, dsradipine, nifedipine, nimodipine, diltiazem, verapamil); Ace inhibitors (e.g., captopril, enalapril, fosinopril, lisinopril, perindopril, quinapril, ramipril, benzapril); Angiotensin II receptor antagonists (e.g., candesartan, irbesartan, losartan, telmisartan, valsartan); Aldosterone antagonists such as spironolactone; centrally acting adrenergic drugs (e.g., clonidine, guanabenz, methyldopa); antiarrhythmic drug of Class I that interfere with the sodium channel (e.g., quinidine, procainamide, disodyramide, lidocaine, mexiletine, tocamide, phenyloin, encamide, flecamide, moricizine, propafenone), Class II that are beta blockers (e.g., esmolol, propranolol, metoprolol); Class III that affect potassium efflux (e.g., amiodarone, azimilide, bretylium, clorilium, dofetilide, tedisamil, ibutilide, sematilide, sotalol); Class IV that affect the AVnode (e.g., verapamil, diltiazem); Class V unknown mechanisms (e.g., adenoide, digoxin); antioxidant drugs such as Vitamin A, vitamin C, vitamin E, Coenzyme Q10, melanonin, carotenoid terpenoids, non-carotenoid terpenoids, flavonoid polyphenolic; antidepressants (e.g., mirtazapine, trazodone); antipsychotic drugs (e.g., fluphenazine, haloperidol, thiotixene, trifluoroperazine, loxapine, perphenazine, clozapine, quetiapine, risperidone, olanzapine); anxyolitics (Benzodiazepines such as diazepam, clonazepam, alprazolam, temazepam, chlordiazepoxide, flunitrazepam, lorazepam, clorazepam; Imidaxopyridines such as Zolpidem, alpidem; Pyrazolopyrimidines such as zaleplon); antiemetic drugs such as Serotonine receptor antagonists (dolasetron, granisetron, ondansetron), dopamine antagonists (domperidone, droperidol, haloperidol, chlorpromazine, promethazine, metoclopramide) antihystamines (cyclizine, diphenydramine, dimenhydrinate, meclizine, promethazine, hydroxyzine); antiinfectives; antihystamines (e.g., mepyramine, antazoline, diphenhydramine, carbinoxamine, doxylamine, clemastine, dimethydrinate, cyclizine, chlorcyclizine, hydroxyzine, meclizine, promethazine, cyprotheptadine, azatidine, ketotifen, acrivastina, loratadine, terfenadine, cetrizidinem, azelastine, levocabastine, olopatadine, levocetrizine, desloratadine, fexofenadine, cromoglicate nedocromil, thiperamide, impromidine); antifungus (e.g., Nystatin, amphotericin B, natamycin, rimocidin, filipin, pimaricin, miconazole, ketoconazole, clotrimazole, econazole, mebendazole, bifonazole, oxiconazole, sertaconazole, sulconazole, tiaconazole, fluconazole, itraconazole, posaconazole, voriconazole, terbinafine, amorolfine, butenafine, anidulafungin, caspofungin, flucytosine, griseofulvin, fluocinonide) and antiviral drugs such as Anti-herpesvirus agents (e.g., Acyclovir, Cidofovir, Docosanol, Famcyclovir, Fomivirsen, Foscarnet, Ganciclovir, Idoxuridine, Penciclovir, Trifluridine, Tromantadine, Valacyclovir, Valgancyclovir, Vidarabine); Anti-influenza agents (Amantadine, Oseltamivir, Peramivir, Rimantadine, Zanamivir); Antiretroviral drugs (abacavir, didanosine, emtricitabine, lamivudine, stavudine, tenofovir, zalcitabine, zidovudine, adeforvir, tenofovir, efavirenz, delavirdine, nevirapine, amprenavir, atazanavir, darunavir, fosamprenavir, indinavir, lopinavir, nelfinavir, ritonavir, saquinavir, tipranavir); other antiviral agents (Enfuvirtide, Fomivirsen, Imiquimod, Inosine, Interferon, Podophyllotoxin, Ribavirin, Viramidine); drugs against neurological dysfunctions such as Parkinson's disease (e.g., dopamine agonists, L-dopa, Carbidopa, benzerazide, bromocriptine, pergolide, pramipexole, ropinipole, apomorphine, lisuride); drugs for the treatment of alcoholism (e.g., antabuse, naltrexone, vivitrol), and other addiction forms; vasodilators for the treatment of erectile dysfunction (e.g., Sildenafil, vardenafil, tadalafil), muscle relaxants (e.g., benzodiazepines, methocarbamol, baclofen, carisoprodol, chlorzoxazone, cyclobenzaprine, dantrolene, metaxalone, orphenadrine, tizanidine); muscle contractors; opioids; stimulating drugs (e.g., amphetamine, cocaine, caffeine, nicotine); tranquillizers; antibiotics such as macrolides; aminoglycosides; fluoroquinolones and beta-lactams; vaccines; cytokines; growth factors; hormones including birth-control drugs; sympathomimetic drugs (e.g., amphetamine, benzylpiperazine, cathinone, chlorphentermine, clobenzolex, cocaine, cyclopentamine, ephedrine, fenfluramine, methylone, methylphenidate, Pemoline, phendimetrazine, phentermine, phenylephrine, propylhexedrine, pseudoephedrine, sibutramine, symephrine); diuretics; lipid regulator agents; antiandrogen agents (e.g., bicalutamide, cyproterone, flutamide, nilutamide); antiparasitics; blood thinners (e.g., warfarin); neoplastic drugs; antineoplastic drugs (e.g., chlorambucil, chloromethine, cyclophosphamide, melphalan, carmustine, fotemustine, lomustine, carboplatin, busulfan, dacarbazine, procarbazine, thioTEPA, uramustine, mechloretamine, methotrexate, cladribine, clofarabine, fludarabine, mercaptopurine, fluorouracil, vinblastine, vincristine, daunorubicin, epirubicin, bleomycin, hydroxyurea, alemtuzumar, cetuximab, aminolevulinic acid, altretamine, amsacrine, anagrelide, pentostatin, tretinoin); hypoglicemics; nutritive and integrator agents; growth integrators; antienteric drugs; vaccines; antibodies; diagnosis and radio-opaque agents; or mixtures of the above mentioned drugs (e.g., combinations for the treatment of asthma containing steroids and beta-agonists); or any other biologically active agent such as growth factors and hormones.

In the presently disclosed co-assembling peptides, the N-terminus and the C-terminus of the peptides may be “capped” by protecting groups such as an acetyl on the N-terminus and an amide on the C-terminus. In other embodiments, the capping groups include, but are not limited to, alkyls (e.g., methyl, alkanes, alkenes, alkynes), arenes (alkyl benzene), aldehydes, ketones, alkyl halides (or halolakanes) and acid halides with halogens (F, Cl, Br, I), alkali metals (Li, Na, K), and alkali earths (Be, Mg, Ca, Sr), alcohols, hydroxyl, ethers, esters, epoxides, nitrate, nitrite, nitrile, nitro, nitroso, imine, imide, azide, cyanide, isocyanide, azo compounds, thiol, sulfide, disulfide, sulfide, sulfoxide, sulfone, sulfinic acid, sulfonic acid, sulfonate ester, thiocyanate, isothiocyanate, thial, thiketone, and phosphine.

In certain embodiments, the present disclosure is directed to a peptide composition comprising anionic peptides comprising Formula 1 and cationic peptides comprising Formula 2, wherein Formula 1 is X_{Ap}X₁X₁ₙX₂X₂ₘX_{Aq} (SEQ ID NO:1) and Formula 2 is X_{Cp}X₁X₁ₙX₂X₂ₘX_{Cq} (SEQ ID NO:2), wherein X_{A} is a negatively charged (anionic) amino acid, X_{C} is a positively charged (cationic) amino acid, X₁ is a phenylalanine or tryptophan, or an analog or derivative of phenylalanine or tryptophan having pi-pi stacking properties, X₂ is a natural or non-natural amino acid, p=0-10, q=0-10, n=1-10, and m=1-10, and with the proviso that at p+q equals at least 1, and wherein the anionic and cationic peptides are able to self-assemble into a peptide nanofiber when the peptide composition is subjected to a self-assembly-stimulating condition.

In certain other embodiments, the present disclosure is directed to a peptide composition comprising anionic peptides comprising Formula 3 and cationic peptides comprising Formula 4, wherein Formula 3 is X_{Ap}X₂X₂ₘX₁X₁ₙX_{Aq} (SEQ ID NO:3) and Formula 4 is X_{Cp}X₂X₂ₘX₁X₁ₙX_{Cq} (SEQ ID NO:4), wherein X_{A} is a negatively charged (anionic) amino acid, X_{C} is a positively charged (cationic) amino acid, X₁ is a phenylalanine or tryptophan, or an analog or derivative of phenylalanine or tryptophan having pi-pi stacking properties, X₂ is a natural or non-natural amino acid, p=0-10, q=0-10, and n=1-10, and m=1-10, and with the proviso that at p+q equals at least 1, and wherein the anionic and cationic peptides are able to self-assemble into a peptide nanofiber when the peptide composition is subjected to a self-assembly-stimulating condition.

In certain other embodiments, the present disclosure is directed to a peptide composition comprising anionic peptides comprising Formula 5 and cationic peptides comprising Formula 6, wherein: Formula 5 is X_{Ap}X₁X₂(X₁X₂)ₙX_{Aq} (SEQ ID NO:5) and Formula 6 is X_{Cp}X₁X₂(X₁X₂)ₙX_{Cq} (SEQ ID NO:)6 wherein X_{A} is a negatively charged (anionic) amino acid, X_{C} is a positively charged (cationic) amino acid, X₁ is a phenylalanine or tryptophan, or an analog or derivative of phenylalanine or tryptophan having pi-pi stacking properties, X₂ is a natural or non-natural amino acid, p=0-10, q=0-10, n=1-10, and with the proviso that at p+q equals at least 1, and wherein the anionic and cationic peptides are able to self-assemble into a peptide nanofiber when the peptide composition is subjected to a self-assembly-stimulating condition.

In certain other embodiments, the present disclosure is directed to a peptide composition comprising anionic peptides comprising Formula 7 and cationic peptides comprising Formula 8, wherein: Formula 7 is X_{Ap}X₂X₁(X₂X₁)ₙX_{Aq} (SEQ ID NO:7) and Formula 8 is X_{Cp}X₂X₁(X₂X₁)ₙX_{Cq} (SEQ ID NO:8), wherein X_{A} is a negatively charged (anionic) amino acid, X_{C} is a positively charged (cationic) amino acid, X₁ is a phenylalanine or tryptophan, or an analog or derivative of phenylalanine or tryptophan having pi-pi stacking properties, X₂ is a natural or non-natural amino acid, p=0-10, q=0-10, n=1-10, and with the proviso that at p+q equals at least 1, and wherein the anionic and cationic peptides are able to self-assemble into a peptide nanofiber when the peptide composition is subjected to a self-assembly-stimulating condition.

In certain non-limiting embodiments of the peptide compositions described above in regard to Formulas 1-8, X_{A} is selected from L- or D-forms of aspartic acid (D) and glutamic acid (E), X_{C} is selected from L- or D-forms of lysine (K), arginine (R), and histidine (H) (L- or D-forms), X₁ is selected from L- or D-forms of phenylalanine (F) or tryptophan (W), and analogs or derivatives thereof that have pi-pi stacking properties, and X₂ is selected from L- or D-forms of glycine (G), alanine (A), aspartic acid, glutamic acid, arginine, lysine, histidine, leucine (L), isoleucine (I), valine (V), serine (S), threonine (T), tyrosine (Y), phenylalanine, tryptophan, methionine (M), cysteine (C), asparagine (N), glutamine (Q), and proline (P) ( ). In certain embodiments of the peptide compositions described above, X_{A} is a non-natural amino acid having a negative charge. In certain embodiments of the peptide compositions described above, X_{c} is a non-natural amino acid having a positive charge. In certain embodiments of the peptide compositions described above, at least one of X_{A}, X_{C}, X₁, and X₂ is a D-amino acid. In certain embodiments of the peptide compositions described above, at least one of the N-terminus and C-terminus of the anionic peptide and/or the cationic peptide is linked to a cargo molecule. In certain embodiments of the peptide compositions described above, each N-terminal X_{A} and X_{C} and each C-terminal X_{A} and X_{C} is covalently linked to a capping group. In certain embodiments of the peptide compositions described above, the capping group linked to each N-terminal X_{A} and X_{C} is an acetyl and the capping group linked to each C-terminal X_{A} and X_{C} is an amide. In certain embodiments of the peptide compositions described above, each anionic and cationic peptide comprises a length in a range of 5 to 42 amino acids. In certain embodiments of the peptide compositions described above, the self-assembly-stimulating condition comprises a pH ranging from about 6.5 to about 8.5. In certain embodiments of the peptide compositions described above, X₂ is a hydrophobic amino acid. For example, the hydrophobic amino acid may be selected from L- or D-forms of glycine, alanine, leucine, isoleucine, valine, phenylalanine, tryptophan, methionine, and proline. In certain embodiments, instead of phenylalanine or tryptophan, X₁ is a non-natural amino acid having pi-pi stacking properties, such as analogs or derivatives of phenylalanine or tryptophan (L- or D-forms). In certain embodiments, the present disclosure is directed to a hydrogel comprising nanofibers constructed from one or more of the self-assembling peptides described herein.

In certain embodiments, the presently disclosed peptide compositions and nanofibers formed therefrom can be used in cancer treatments. For example, the nanofibers can be internalized by cancer cells and can cause significant cell death in very low concentrations in a very short time (e.g., 6 h). The nanofibers are not cytotoxic to normal cells, which proliferate over a longer duration. In certain other embodiments, the disclosed peptide compositions can be used as vaccine platforms. Individual peptides of the co-assembly pairs can be functionalized with different antigen epitopes. The nanofibers formed therefrom display these epitopes in an ordered array, which can trigger the immune response efficiently. For example, nanofibers were self-assembled from peptides to which large 55 kDa hydrophobic proteins (ovalbumin) were attached. In another embodiment, the nanofibers formed from the presently disclosed self-assembling peptides can have adjuvant activity (immune stimulation), either as a self-adjuvant when the nanofibers is a vaccine scaffold itself, or as an adjuvant in other vaccine formulations. Moreover, such behavior in the tumor tissue can initiate more efficient immune response and a better immunotherapy as the tumor specific proteins can be uptake with higher yield to the immune cells.

In another embodiment, the nanofibers formed from the presently disclosed self-assembling peptides can be used as a bacterial cancer therapy. For example, tumor-localized bacteria can produce certain embodiments of the presently disclosed peptides in the core of the tumor. Moreover, the peptides can cause necrosis in the tumor cells and initiate a high immune response. The release of bacterial residues from the tumor can also recruit immune cells and behave as a self-adjuvant and amplify the immune response.

In another embodiment, the nanofibers formed from the presently disclosed self-assembling peptides can be used to form a hydrogel which can be used, for example, as a scaffold for tissue engineering. As described before, individual peptides can be functionalized with variety of proteins and peptides. In certain embodiments, hydrogels comprising the presently disclosed nanofibers can recruit specific cells, such as specific immune cells. As a result, in one embodiment, the hydrogel can work as an artificial lymph node. The mechanical properties of the hydrogels can be tuned (altered) by using particular amino acids in the “X₂” positions of the peptides for desired tissue engineering applications. In certain embodiments, the self-assembly of the anionic and cationic peptides into nanofibers is carried out in a medium in which cells are being cultured. In other embodiments, the nanofibers can be formed with metals or other inorganic components to form organic-inorganic nanofibers which can be used for scaffold construction.

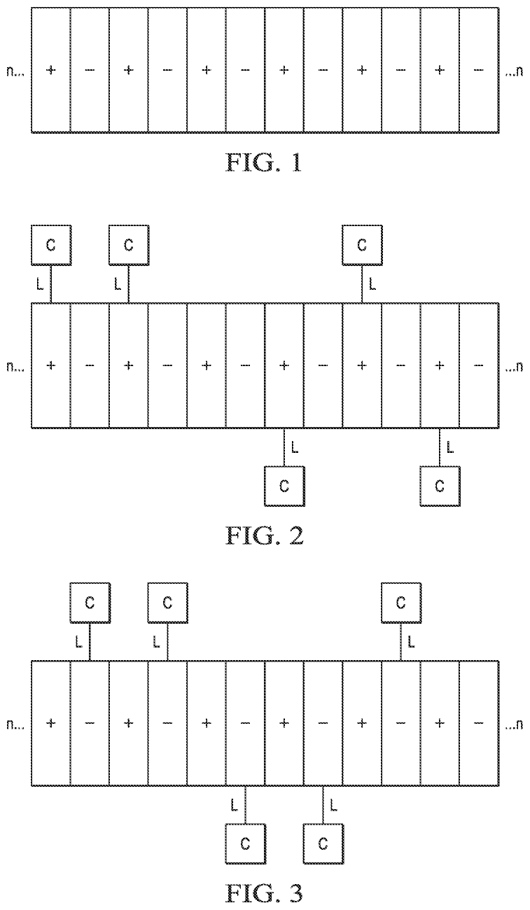

Shown schematically in FIG. 1 is a representation of a nanostructure (i.e., nanofiber) which results from the co-assembly of the complementarily-charged peptide segments disclosed herein. The peptide segments do not assemble until exposed to suitable conditions, such as a neutral pH and/or a near physiologic pH. In some embodiments the neutral and/or near physiologic pH can range from about 6.5 to about 8.5, or from about 6.5 to about 7.5. The charge of the complementary peptide segments can be either positive or negative, which refers to the net charge (cationic or anionic) of the entire peptide segment.

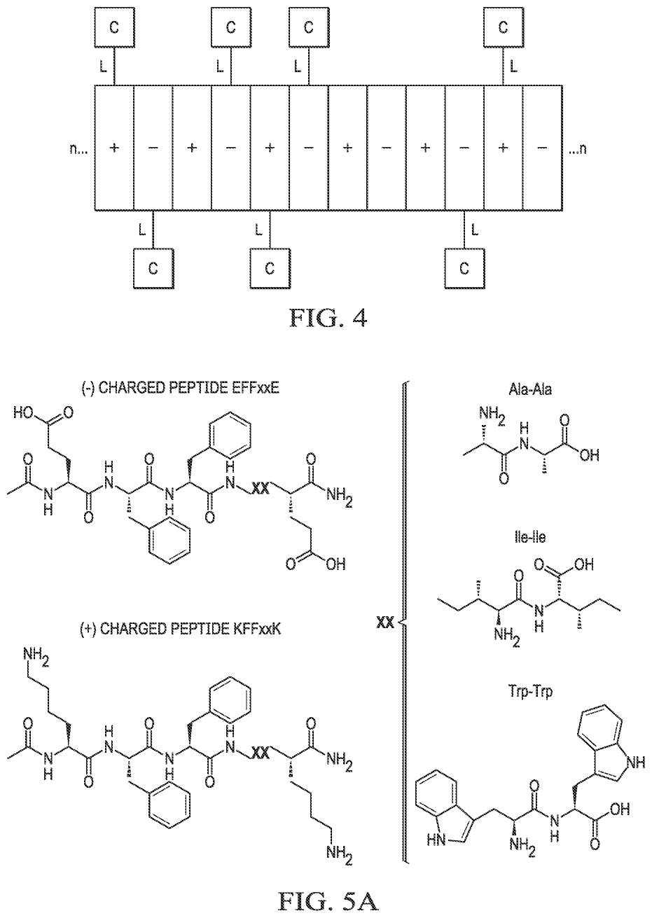

As shown in FIGS. 2-4, the nanofibers formed from the self-assembly of the anionic and cationic peptide segments can be modified to carry cargo molecules C coupled directly or indirectly (via linkers L) to the anionic and/or cationic peptide segments, or can be formed from anionic and cationic peptide segments modified to carry cargo molecules before the peptide segments are combined to form the nanofibers.

Table 1A shows examples of exemplary, non-limiting, anionic-cationic peptide pairs of the present disclosure which are based on Formulas 1 and 2 where X_{A}=D or E (L- or D-forms); X_{C}=K, R or H (L- or D-forms); X₁=F (L- or D-forms); X₂ is a natural amino acid (or a D-form thereof); and p, q, n, and m=1.

TABLE 1A
Exemplary anionic and cationic peptide pairs
based on Formulas 1 and 2 where X₁ = F*;
X₂ is a natural amino acid*; and p, q, n,
and m = 1.

| | Anionic peptide, | Cationic peptide, |
|---|---|---|
| | X_{A} = E or D* | X_{C} = K, R, or H* |
| X₂ | (SEQ ID NO:) | (SEQ ID NO:) |
| A | X_{A}FFAAX_{A} (9) | X_{C}FFAAX_{C} (10) |
| W | X_{A}FFWWX_{A} (11) | X_{C}FFWWX_{C} (12) |
| I | X_{A}FFIIX_{A} (13) | X_{C}FFIIX_{C} (14) |
| F | X_{A}FFFFX_{A} (15) | X_{C}FFFFX_{C} (16) |
| V | X_{A}FFVVX_{A} (17) | X_{C}FFVVX_{C} (18) |
| L | X_{A}FFLLX_{A} (19) | X_{C}FFLLX_{C} (20) |
| G | X_{A}FFGGX_{A} (21) | X_{C}FFGGX_{C} (22) |
| C | X_{A}FFCCX_{A} (23) | X_{C}FFCCX_{C} (24) |
| D | X_{A}FFDDX_{A} (25) | X_{C}FFDDX_{C} (26) |
| E | X_{A}FFEEX_{A} (27) | X_{C}FFEEX_{C} (28) |
| S | X_{A}FFSSX_{A} (29) | X_{C}FFSSX_{C} (30) |
| H | X_{A}FFHHX_{A} (31) | X_{C}FFHHX_{C} (32) |
| Y | X_{A}FFYYX_{A} (33) | X_{C}FFYYX_{C} (34) |
| K | X_{A}FFKKX_{A} (35) | X_{C}FFKKX_{C} (36) |
| R | X_{A}FFRRX_{A} (37) | X_{C}FFRRX_{C} (38) |
| N | X_{A}FFNNX_{A} (39) | X_{C}FFNNX_{C} (40) |
| Q | X_{A}FFQQX_{A} (41) | X_{C}FFQQX_{C} (42) |
| M | X_{A}FFMMX_{A} (43) | X_{C}FFMMX_{C} (44) |
| P | X_{A}FFPPX_{A} (45) | X_{C}FFPPX_{C} (46) |
| T | X_{A}FFTTX_{A} (47) | X_{C}FFTTX_{C} (48) |

*D- or L- forms

Table 1B shows examples of exemplary, non-limiting, anionic-cationic peptide pairs of the present disclosure which are based on Formulas 1 and 2 where X_{A}=D or E (L- or D-forms); X_{C}=K, R or H (L- or D-forms); X₁=W (L- or D-forms); X₂ is a natural amino acid (or a D-amino acid form thereof); and p, q, n, and m=1.

TABLE 2B
Exemplary anionic and cationic peptide pairs
based on Formulas 1 and 2 where X₁ = W*;
X₂ is a natural amino acid*; and p, q, n, and
m = 1.

| | Anionic peptide, | Cationic peptide, |
|---|---|---|
| | X_{A} = E or D* | X_{C} = K, R, or H* |
| X₂ | (SEQ ID NO:) | (SEQ ID NO:) |
| A | X_{A}WWAAX_{A} (49) | X_{C}WWAAX_{C} (50) |
| W | X_{A}WWWWX_{A} (51) | X_{C}WWWWX_{C} (52) |
| I | X_{A}WWIIX_{A} (53) | X_{C}WWIIX_{C} (54) |
| F | X_{A}WWFFX_{A} (55) | X_{C}WWFFX_{C} (56) |
| V | X_{A}WWVVX_{A} (57) | X_{C}WWVVX_{C} (58) |
| L | X_{A}WWLLX_{A} (59) | X_{C}WWLLX_{C} (60) |
| G | X_{A}WWGGX_{A} (61) | X_{C}WWGGX_{C} (62) |
| C | X_{A}WWCCX_{A} (63) | X_{C}WWCCX_{C} (64) |
| D | X_{A}WWDDX_{A} (65) | X_{C}WWDDX_{C} (66) |
| E | X_{A}WWEEX_{A} (67) | X_{C}WWEEX_{C} (68) |
| S | X_{A}WWSSX_{A} (69) | X_{C}WWSSX_{C} (70) |
| H | X_{A}WWHHX_{A} (71) | X_{C}WWHHX_{C} (72) |
| Y | X_{A}WWYYX_{A} (73) | X_{C}WWYYX_{C} (74) |
| K | X_{A}WWKKX_{A} (75) | X_{C}WWKKX_{C} (76) |
| R | X_{A}WWRRX_{A} (77) | X_{C}WWRRX_{C} (78) |
| N | X_{A}WWNNX_{A} (79) | X_{C}WWNNX_{C} (70) |
| Q | X_{A}WWQQX_{A} (71) | X_{C}WWQQX_{C} (72) |
| M | X_{A}WWMMX_{A} (73) | X_{C}WWMMX_{C} (74) |
| P | X_{A}WWPPX_{A} (75) | X_{C}WWPPX_{C} (76) |
| T | X_{A}WWTTX_{A} (77) | X_{C}WWTTX_{C} (78) |

*D- or L- forms

Table 2A shows examples of exemplary, non-limiting, anionic-cationic peptide pairs of the present disclosure which are based on Formulas 3 and 4 where X_{A}=D or E (L- or D-forms); X_{C}=K, R or H (L- or D-forms); X₁=F (L- or D-forms); X₂ is a natural amino acid (or a D-amino acid form thereof); and p, q, n, and m=1.

TABLE 2A
Exemplary anionic and cationic peptide pairs
based on Formulas 3 and 4 where X₁ = F*;
X₂ is a natural amino acid*; and p, q, n,
and m = 1.

| | Anionic peptide, | Cationic peptide, |
|---|---|---|
| | X_{A} = E or D* | X_{C} = K, R, or H* |
| X₂ | (SEQ ID NO:) | (SEQ ID NO:) |
| A | X_{A}AAFFX_{A} (79) | X_{C}AAFFX_{C} (80) |
| W | X_{A}WWFFX_{A} (81) | X_{C}WWFFX_{C} (82) |
| I | X_{A}IIFFX_{A} (83) | X_{C}IIFFX_{C} (84) |
| V | X_{A}VVFFX_{A} (85) | X_{C}VVFFX_{C} (86) |
| L | X_{A}LLFFX_{A} (87) | X_{C}LLFFX_{C} (88) |
| G | X_{A}GGFFX_{A} (89) | X_{C}GGFFX_{C} (90) |
| C | X_{A}CCFFX_{A} (91) | X_{C}CCFFX_{C} (92) |
| D | X_{A}DDFFX_{A} (93) | X_{C}DDFFX_{C} (94) |
| E | X_{A}EEFFX_{A} (95) | X_{C}EEFFX_{C} (96) |
| S | X_{A}SSFFX_{A} (97) | X_{C}SSFFX_{C} (98) |
| H | X_{A}HHFFX_{A} (99) | X_{C}HHFFX_{C} (100) |
| Y | X_{A}YYFFX_{A} (101) | X_{C}YYFFX_{C} (102) |
| K | X_{A}KKFFX_{A} (103) | X_{C}KKFFX_{C} (104) |
| R | X_{A}RRFFX_{A} (105) | X_{C}RRFFX_{C} (106) |
| N | X_{A}NNFFX_{A} (107) | X_{C}NNFFX_{C} (108) |
| Q | X_{A}QQFFX_{A} (109) | X_{C}QQFFX_{C} (110) |
| M | X_{A}MMFFX_{A} (111) | X_{C}MMFFX_{C} (112) |
| P | X_{A}PPFFX_{A} (113) | X_{C}PPFFX_{C} (114) |
| T | X_{A}TTFFX_{A} (115) | X_{C}TTFFX_{C} (116) |

*D- or L- forms

Table 2B shows examples of exemplary, non-limiting, anionic-cationic peptide pairs of the present disclosure which are based on Formulas 3 and 4 where X_{A}=D or E (L- or D-forms); X_{C}=K, R or H (L- or D-forms); X₁=W (L- or D-forms); X₂ is a natural amino acid (or a D-amino acid form thereof); and p, q, n, and m=1.

TABLE 2B
Exemplary anionic and cationic peptide pairs
based on Formulas 3 and 4 where X₁ = W*;
X₂ is a natural amino acid*; and p, q, n,
and m = 1.

| | Anionic peptide, | Cationic peptide, |
|---|---|---|
| | X_{A} = E or D* | X_{C} = K, R, or H* |
| X₂ | (SEQ ID NO:) | (SEQ ID NO:) |
| A | X_{A}AAWWX_{A} (117) | X_{C}AAWWX_{C} (118) |
| I | X_{A}IIWWX_{A} (119) | X_{C}IIWWX_{C} (120) |
| V | X_{A}VVWWX_{A} (121) | X_{C}VVWWX_{C} (122) |
| L | X_{A}LLWWX_{A} (123) | X_{C}LLWWX_{C} (124) |
| G | X_{A}GGWWX_{A} (125) | X_{C}GGWWX_{C} (126) |
| C | X_{A}CCWWX_{A} (127) | X_{C}CCWWX_{C} (128) |
| D | X_{A}DDWWX_{A} (129) | X_{C}DDWWX_{C} (130) |
| E | X_{A}EEWWX_{A} (131) | X_{C}EEWWX_{C} (132) |
| S | X_{A}SSWWX_{A} (133) | X_{C}SSWWX_{C} (134) |
| H | X_{A}HHWWX_{A} (135) | X_{C}HHWWX_{C} (136) |
| Y | X_{A}YYWWX_{A} (137) | X_{C}YYWWX_{C} (138) |
| K | X_{A}KKWWX_{A} (139) | X_{C}KKWWX_{C} (140) |
| R | X_{A}RRWWX_{A} (141) | X_{C}RRWWX_{C} (142) |
| N | X_{A}NNWWX_{A} (143) | X_{C}NNWWX_{C} (144) |
| Q | X_{A}QQWWX_{A} (145) | X_{C}QQWWX_{C} (146) |
| M | X_{A}MMWWX_{A} (147) | X_{C}MMWWX_{C} (148) |
| P | X_{A}PPWWX_{A} (149) | X_{C}PPWWX_{C} (150) |
| T | X_{A}TTWWX_{A} (151) | X_{C}TTWWX_{C} (152) |

* D- or L- forms

Table 3A shows examples of exemplary, non-limiting, anionic-cationic peptide pairs of the present disclosure which are based on Formulas 5 and 6 where X_{A}=D or E (L- or D-forms); X_{C}=K, R or H (L- or D-forms); X₁=F (L- or D-forms); X₂ is a natural amino acid (or a D-amino acid form thereof); and p, q, and n=1.

TABLE 3A
Exemplary anionic and cationic peptide pairs
based on Formulas 5 and 6 where X₁ = F*;
X₂ is a natural amino acid*; and p, q,
and n = 1.

| | Anionic peptide, | Cationic peptide, |
|---|---|---|
| | X_{A} = E or D* | X_{C} = K, R, or H* |
| X₂ | (SEQ ID NO:) | (SEQ ID NO:) |
| A | X_{A}FAFAX_{A} (153) | X_{C}FAFAX_{C} (154) |
| W | X_{A}FWFWX_{A} (155) | X_{C}FWFWX_{C} (156) |
| I | X_{A}FIFIX_{A} (157) | X_{C}FIFIX_{C} (158) |
| V | X_{A}FVFVX_{A} (159) | X_{C}FVFVX_{C} (160) |
| L | X_{A}FLFLX_{A} (161) | X_{C}FLFLX_{C} (162) |
| G | X_{A}FGFGX_{A} (163) | X_{C}FGFGX_{C} (164) |
| C | X_{A}FCFCX_{A} (165) | X_{C}FCFCX_{C} (166) |
| D | X_{A}FDFDX_{A} (167) | X_{C}FDFDX_{C} (168) |
| E | X_{A}FEFEX_{A} (169) | X_{C}FEFEX_{C} (170) |
| S | X_{A}FSFSX_{A} (171) | X_{C}FSFSX_{C} (172) |
| H | X_{A}FHFHX_{A} (173) | X_{C}FHFHX_{C} (174) |
| Y | X_{A}FYFYX_{A} (175) | X_{C}FYFYX_{C} (176) |
| K | X_{A}FKFKX_{A} (177) | X_{C}FKFKX_{C} (178) |
| R | X_{A}FRFRX_{A} (179) | X_{C}FRFRX_{C} (180) |
| N | X_{A}FNFNX_{A} (181) | X_{C}FNFNX_{C} (182) |
| Q | X_{A}FQFQX_{A} (183) | X_{C}FQFQX_{C} (184) |
| M | X_{A}FMFMX_{A} (185) | X_{C}FMFMX_{C} (186) |
| P | X_{A}FPFPX_{A} (187) | X_{C}FPFPX_{C} (188) |
| T | X_{A}FTFTX_{A} (189) | X_{C}FTFTX_{C} (190) |

* D- or L- forms

Table 3B shows examples of exemplary, non-limiting, anionic-cationic peptide pairs of the present disclosure which are based on Formulas 5 and 6 where X_{A}=D or E (L- or D-forms); X_{C}=K, R or H (L- or D-forms); X₁=W (L- or D-forms); X₂ is a natural amino acid (or a D-amino acid form thereof); and p, q, and n=1.

TABLE 3B
Exemplary anionic and cationic peptide pairs
based on Formulas 5 and 6 where X₁ = W*;
X₂ is a natural amino acid*; and p, q,
and n = 1.

| | Anionic peptide, | Cationic peptide, |
|---|---|---|
| | X_{A} = E or D* | X_{C} = K, R, or H* |
| X₂ | (SEQ ID NO:) | (SEQ ID NO:) |
| A | X_{A}WAWAX_{A} (191) | X_{C}WAWAX_{C} (192) |
| I | X_{A}WIWIX_{A} (193) | X_{C}WIWIX_{C} (194) |
| F | X_{A}WFWFX_{A} (195) | X_{C}WFWFX_{C} (196) |
| V | X_{A}WVWVX_{A} (197) | X_{C}WVWVX_{C} (198) |
| L | X_{A}WLWLX_{A} (199) | X_{C}WLWLX_{C} (200) |
| G | X_{A}WGWGX_{A} (201) | X_{C}WGWGX_{C} (202) |
| C | X_{A}WCWCX_{A} (203) | X_{C}WCWCX_{C} (204) |
| D | X_{A}WDWDX_{A} (205) | X_{C}WDWDX_{C} (206) |
| E | X_{A}WEWEX_{A} (207) | X_{C}WEWEX_{C} (208) |
| S | X_{A}WSWSX_{A} (209) | X_{C}WSWSX_{C} (210) |
| H | X_{A}WHWHX_{A} (211) | X_{C}WHWHX_{C} (212) |
| Y | X_{A}WYWYX_{A} (213) | X_{C}WYWYX_{C} (214) |
| K | X_{A}WKWKX_{A} (215) | X_{C}WKWKX_{C} (216) |
| R | X_{A}WRWRX_{A} (217) | X_{C}WRWRX_{C} (218) |
| N | X_{A}WNWNX_{A} (219) | X_{C}WNWNX_{C} (220) |
| Q | X_{A}WQWQX_{A} (221) | X_{C}WQWQX_{C} (222) |
| M | X_{A}WMWMX_{A} (223) | X_{C}WMWMX_{C} (224) |
| P | X_{A}WPWPX_{A} (225) | X_{C}WPWPX_{C} (226) |
| T | X_{A}WTWTX_{A} (227) | X_{C}WTWTX_{C} (228) |

*D- or L- forms

Table 4A shows examples of exemplary, non-limiting, anionic-cationic peptide pairs of the present disclosure which are based on Formulas 7 and 8 where X_{A}=D or E (L- or D-forms); X_{C}=K, R or H (L- or D-forms); X₁=F (L- or D-forms); X₂ is a natural amino acid (or a D-amino acid form thereof); and p, q, and n=1.

TABLE 4A
Exemplary anionic and cationic peptide pairs
based on Formulas 7 and 8 where X₁ = F*;
X₂ is a natural amino acid*; and p, q,
and n = 1.

| | Anionic peptide, | Cationic peptide, |
|---|---|---|
| | X_{A} = E or D* | X_{C} = K, R, or H* |
| X₂ | (SEQ ID NO:) | (SEQ ID NO:) |
| A | X_{A}AFAFX_{A} (229) | X_{C}AFAFX_{C} (230) |
| I | X_{A}IFIFX_{A} (231) | X_{C}IFIFX_{C} (232) |
| V | X_{A}VFVFX_{A} (233) | X_{C}VFVFX_{C} (234) |
| L | X_{A}LFLFX_{A} (235) | X_{C}LFLFX_{C} (236) |
| G | X_{A}GFGFX_{A} (237) | X_{C}GFGFX_{C} (238) |
| C | X_{A}CFCFX_{A} (239) | X_{C}CFCFX_{C} (240) |
| D | X_{A}DFDFX_{A} (241) | X_{C}DFDFX_{C} (242) |
| E | X_{A}EFEFX_{A} (243) | X_{C}EFEFX_{C} (244) |
| S | X_{A}SFSFX_{A} (245) | X_{C}SFSFX_{C} (246) |
| H | X_{A}HFHFX_{A} (247) | X_{C}HFHFX_{C} (248) |
| Y | X_{A}YFYFX_{A} (249) | X_{C}YFYFX_{C} (250) |
| K | X_{A}KFKFX_{A} (251) | X_{C}KFKFX_{C} (252) |
| R | X_{A}RFRFX_{A} (253) | X_{C}RFRFX_{C} (254) |
| N | X_{A}NFNFX_{A} (255) | X_{C}NFNFX_{C} (256) |
| Q | X_{A}QFQFX_{A} (257) | X_{C}QFQFX_{C} (258) |
| M | X_{A}MFMFX_{A} (259) | X_{C}MFMFX_{C} (260) |
| P | X_{A}PFPFX_{A} (261) | X_{C}PFPFX_{C} (262) |
| T | X_{A}TFTFX_{A} (263) | X_{C}TFTFX_{C} (264) |

* D- or L- forms

Table 4B shows examples of exemplary, non-limiting, anionic-cationic peptide pairs of the present disclosure which are based on Formulas 7 and 8 where X_{A}=D or E (L- or D-forms); X_{C}=K, R or H (L- or D-forms); X₁=W (L- or D-forms); X₂ is a natural amino acid (or a D-amino acid form thereof); and p, q, and n=1.

TABLE 4B
Exemplary anionic and cationic peptide pairs
based on Formulas 7 and 8 where X₁ = W*;
X₂ is a natural amino acid*; and p, q,
and n = 1.

| | Anionic peptide, | Cationic peptide, |
|---|---|---|
| | X_{A} = E or D* | X_{C} = K, R, or H* |
| X₂ | (SEQ ID NO:) | (SEQ ID NO:) |
| A | X_{A}AWAWX_{A} (265) | X_{C}AWAWX_{C} (266) |
| I | X_{A}IWIWX_{A} (267) | X_{C}IWIWX_{C} (268) |
| V | X_{A}VWVWX_{A} (269) | X_{C}VWVWX_{C} (270) |
| L | X_{A}LWLWX_{A} (271) | X_{C}LWLWX_{C} (272) |
| G | X_{A}GWGWX_{A} (273) | X_{C}GWGWX_{C} (274) |
| C | X_{A}CWCWX_{A} (275) | X_{C}CWCWX_{C} (276) |
| D | X_{A}DWDWX_{A} (277) | X_{C}DWDWX_{C} (278) |
| E | X_{A}EWEWX_{A} (279) | X_{C}EWEWX_{C} (280) |
| S | X_{A}SWSWX_{A} (281) | X_{C}SWSWX_{C} (282) |
| H | X_{A}HWHWX_{A} (283) | X_{C}HWHWX_{C} (284) |
| Y | X_{A}YWYWX_{A} (285) | X_{C}YWYWX_{C} (286) |
| K | X_{A}KWKWX_{A} (287) | X_{C}KWKWX_{C} (288) |
| R | X_{A}RWRWX_{A} (289) | X_{C}RWRWX_{C} (290) |
| N | X_{A}NWNWX_{A} (291) | X_{C}NWNWX_{C} (292) |
| Q | X_{A}QWQWX_{A} (293) | X_{C}QWQWX_{C} (294) |
| M | X_{A}MWMWX_{A} (295) | X_{C}MWMWX_{C} (296) |
| P | X_{A}PWPWX_{A} (297) | X_{C}PWPWX_{C} (298) |
| T | X_{A}TWTWX_{A} (299) | X_{C}TWTWX_{C} (300) |

* D- or L- forms

### EXAMPLES

Certain embodiments of the present disclosure will now be discussed in terms of several specific, non-limiting, examples. The examples described below will serve to illustrate the general practice of the present disclosure, it being understood that the particulars shown are merely exemplary for purposes of illustrative discussion of particular embodiments of the present disclosure only and are not intended to be limiting of the claims of the present disclosure.

Methods

9-fluorenylmethoxycarbonyl (Fmoc) protected amino acids, [4-[α-(2′,4′-dimethoxyphenyl) Fmoc aminomethyl] phenoxy] acetamidonorleucyl-MBHA resin (Rink amide MBHA resin), 2-(6-Chloro-1-H-benzotriazole-1-yl)-1,1,3,3-tetramethylaminium hexafluorophosphate (HCTU), 4-Methylmorpholine (NMM), Trifluoroacetic acid (TFA), piperidine, dimethylformamide (DMF), dichloromethane (DCM) were purchased from Gyros Protein Technologies. Triisopropylsilane, Acetic anhydride, Congo Red dye and pyrene were purchased from Sigma-Aldrich. Deionized water (resistance of 18 MΩ·cm) was used during the experiments.

Synthesis and Characterization of FF Peptides.

Anionic-cationic self-assembling peptide pairs (see Table 5) were synthesized by using solid phase peptide synthesis method with PreludeX automatic peptide synthesizer (Protein Technologies, Inc., Tucson, AZ). Peptides were prepared on a 0.2 scale by repeated amino acid couplings using Fmoc protected amino acid (5 eq.), HCTU (4.875 eq.) and NMM (7.5 eq.). BHA Rink Amide resin was used as solid support to construct the peptides. Fmoc protecting group of amino acids except that of final residue was removed through the treatment with 20% piperidine/DMF solution for 10 min two times at 50° C. Cleavage of the peptides from resin and deprotection of acid labile protected amino acids were carried out with a mixture of TFA/TIS/water in a ratio of 95:2.5:2.5 for 2.5 h. Excess TFA and organic solvents were removed by solvent evaporator and remaining peptide was precipitated using diethyl ether at −20° C. overnight. The centrifuged white peptide precipitate was dissolved in water and freeze at −80° C. overnight and then lyophilized (Labconco Freezone, 12 L) for one or two days. Peptides were purified with preparative HPLC (Agilent 1260) and identified by Shimadzu LCMS-2020. Agilent ZORBAX 300 SB-C18 (9.4×250 mm) and Alltech Prosphere HP C4 300 A 5u (250 mm×4.6 mm) with a mobile phase of water/acetonitrile mixture (0.1% ammonium hydroxide) was used for negatively charged peptides and water/acetonitrile mixture (0.1% formic acid) was used for positively charged peptides. All peptides tested with a purity >95%. HPLC run started with 100% water for 3 min, followed by a gradient increase in acetonitrile from 0% to 60% over 30 min, followed 100% acetonitrile for 3 min and finally 100% water for 3 min. Flow rate is 1.5 mL/min and injection volume are 10 L.

TABLE 5
Specific anionic peptide/cationic peptide pairs.

| Peptide | Anionic peptide | Cationic peptide |
|---|---|---|
| pair name | (SEQ ID NO:) | (SEQ ID NO:) |
| [AA] | EFFAAE (301) | KFFAAK (302) |
| [WW] | EFFWWE (303) | KFFWWK (304) |
| [II] | EFFIIE (305) | KFFIIK (306) |
| [FF] | EFFFFE (307) | KFFFFK (308) |
| [VV] | EFFVVE (309) | KFFVVK (310) |
| [LL] | EFFLLE (313) | KFFLLK (314) |
| [GG] | EFFGGE (315) | KFFGGK (316) |

Critical Aggregation Concentration (CAC) Determination.

CAC of the peptides were examined by using pyrene based on previously published procedure with slight modifications.³¹ Aliquots of pyrene solutions (225 μM in acetone, 4 μL) were added to positively charged peptide and then negatively charged peptide was added with a range of concentration. Final pyrene concentration in the system was 4.5 μM in microplates. Solutions were waited for 30 min to reach equilibrium. Excitation was carried out at 334 nm, and emission spectra were recorded ranging from 360 to 600 nm with microplate reader (BioTek Neo2SM). Both excitation and emission bandwidths were 9 nm. From the pyrene emission spectra, the fluorescence intensity ratio of the vibronic bands (I₃₉₇/I₃₈₀) was plotted against the logarithm of the concentration of the self-assembled peptides, and the CAC value was calculated from the intersection of the tangents.

Congo Red Assay.

Congo red assay is used for amyloid fibril detection. In case of the presence of high beta-sheet organization, congo red lies parallel to fibril axis and induces a red shift in the absorption maximum (498 nm). Congo red dye was dissolved in PBS to a final concentration of 500 μM. For sample preparation, stock peptides in PBS (10 mM) diluted to 1 mM in PBS. Then 1 mM of negative peptide (50 μL) added to 96 black well plate, then 1 mM of positively charged peptide (50 μL) added to the same place. 2 μL of 500 μM Congo red is immediately added to the solution and read at microplate reader (BioTek Neo2SM) at room temperature, spectral scanning for absorbance was adjusted between 400-600 nm. The control group of only congo red dye in PBS was also prepared with a final concentration of 10 μM. The analysis was performed for 2 days with 10 time points. Spectral shift was calculated based on congo red only absorption maximum.

Rheology Measurements.

Rheology measurements of 10 mM samples at pH 7 were performed to understand the mechanical properties of the resulting gels. At rheometer stage first negatively charged and then positively charged peptide added, and immediate analysis was performed. Total volume of samples was 200 μL (100 μL positively charged, 100 μL negatively charged peptide). A Discovery Hybrid Rheometer-2 (TA Instruments, New Castle, DE) equipped with parallel 20-mm plate was used for the analysis Measuring distance was determined as 0.5 mm. Time sweep tests of each sample were carried out for 20 min. Angular frequency and strain magnitudes were determined as ω=10 rad/s and γ=0.1%, respectively. In order to determine linear viscoelastic region (LVE), Amplitude sweep tests of 10 mM samples at pH 7 were performed in the same configuration and concentration. Strain value logarithmically increased from 0.1 to 100%, total of 61 value was measured. Angular frequency was kept as constant at ω=10 rad/s. Medium contains 10% FBS and 1% antibiotic with RPMI.

FT-IR Measurements.

10 mM, 20 μL of peptides in different solvent were analyzed with Bruker Tensor II with BioATR II unit. For background spectrum, water, PBS and medium was measured first then peptides with those solvent was subtracted as a background scans automatically in the device. Analysis was performed between 1800 and 900 cm⁻¹ with 4 cm⁻¹ resolution and 30 scans.

NMR Measurements.

NMR spectra were obtained on Varian VNMRS 500 MHz instruments at the NMR facility of the Department of Chemistry and Biochemistry of the University of Oklahoma using 90% D2O. 1H, 13C, and 15N chemical shifts were referenced to internal solvent resonances. Chemical shifts are reported in parts per million (ppm) and coupling constants J are given in Hz. All NMR spectra were recorded at ambient temperature and processed using MestReNova software.

### Example 1

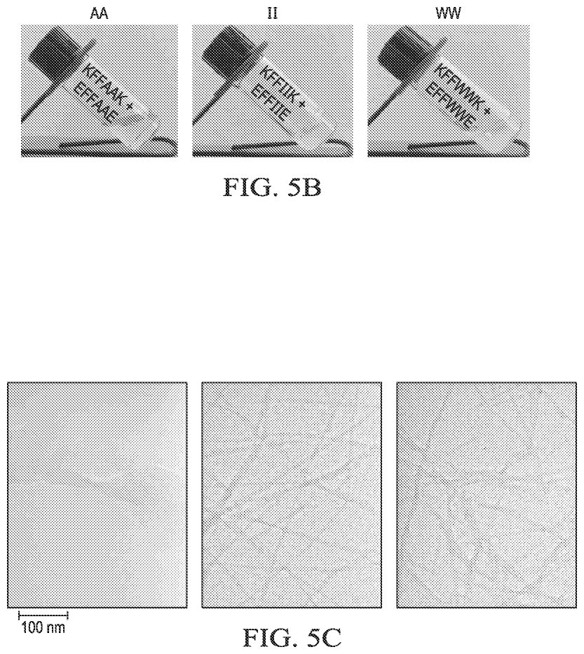

Seven sets of anionic/cationic peptides (designated [AA], [II], [WW], [FF], [VV], [LL], and [GG]) were prepared for individual testing (Table 5). Three sets, [AA], [II], [WW], are shown in FIG. 5A. To preserve the unique charges of the peptides, the N-termini of the peptides were acetylated and the C-termini were amidated. FIGS. 5B-C show results of combining the [AA], [II], and [WW] peptide sets. Upon mixing, anionic/cationic peptides comprising II and WW, respectively, self-assembled into gels (FIG. 5B) comprising liner nanofibers (FIG. 5C). Anionic/cationic peptides comprising AA did not appear to form an organized structure (FIG. 5C).

### Example 2

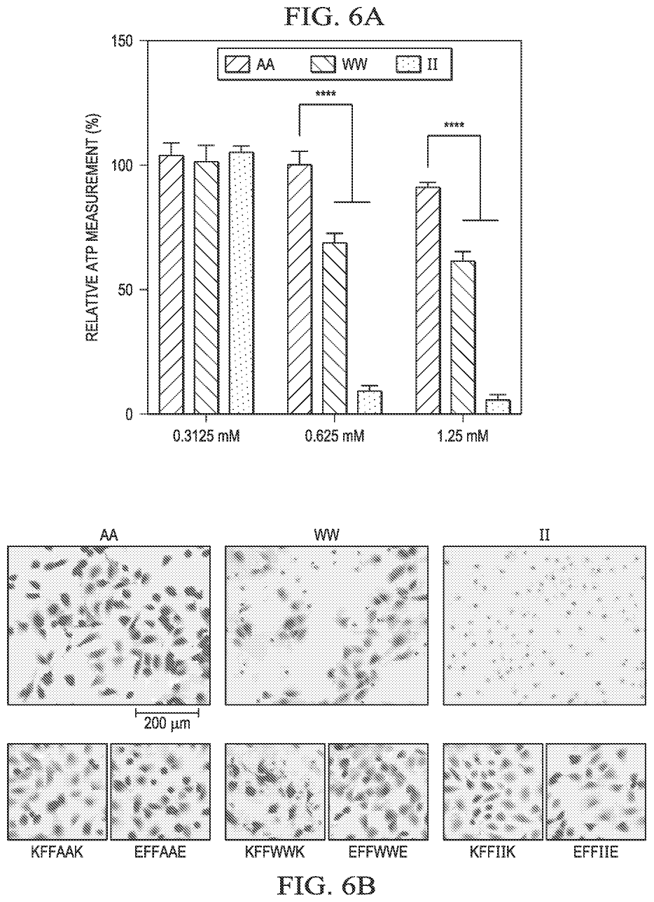

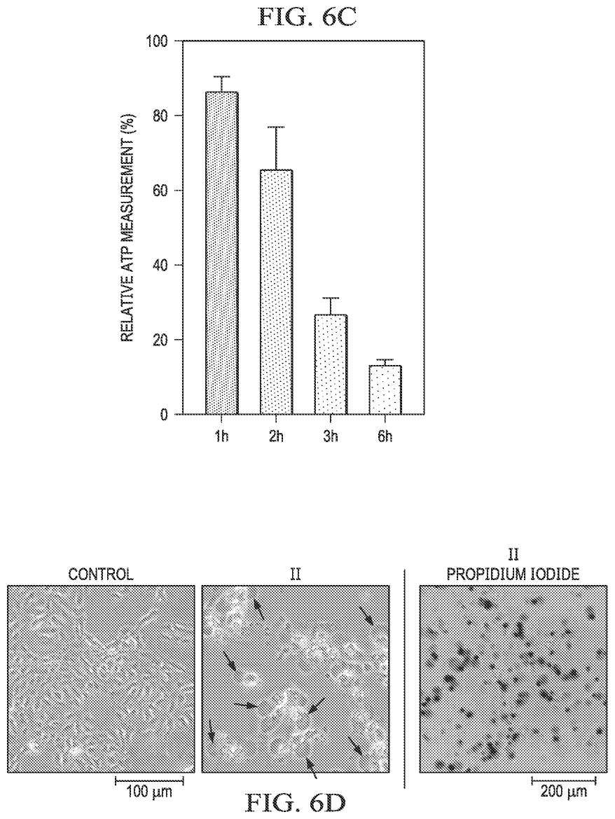

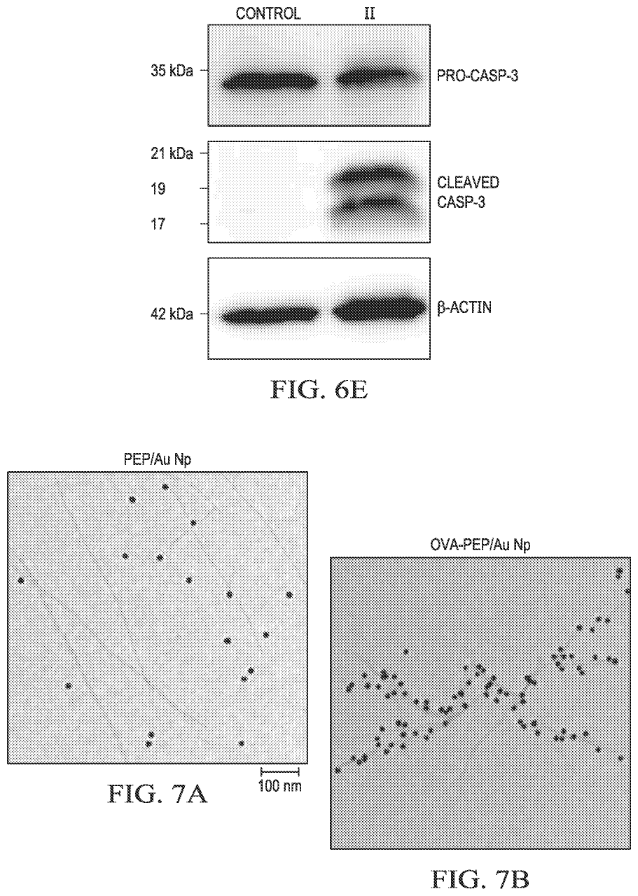

Incubation of the peptides with OVCAR-8 ovarian cancer cells were incubated with the peptides of the [AA], [II], [WW] peptide sets. Except for KFFWWK (SEQ ID NO:304), the individual charged components of the peptide sets did not show any toxicity. The nanofibers formed from the [WW] peptide set did not show any toxicity, which indicates that the formed fibers of [WW] are highly stable. Although, the individual peptide components of [II] did not show any toxicity, the formed [II] nanofibers killed the cells (FIGS. 6A-B). Red fluorescence (ethidium homodimer-1) was associated with loss of plasma membrane integrity whereas the green fluorescence correlated with intracellular esterase activity of metabolically active cells (color not shown in figures). Pyroptosis is a newly identified cell death modality characterized by the formation of large bubbles on the plasma membrane and cell swelling. Recently pyroptosis has been classified as a regulated cell death (RCD) by Nomenclature Committee on Cell Death (NCCD). Peptide-treated OVCAR-8 cells indicated pyroptotic morphology. One of the pathways mediating pyroptosis is through cleavage of caspase-3.

Pyroptosis is shown as the most immunogenic cell death mechanism; genetically engineered cancer cells in tumors on mice showed the highest immune cell attraction upon pyroptosis induction. The immune system was trained with the damaged cancer cell debris and suppressed the tumor formation when the mice were rechallenged. Observation of pyroptosis as a result of incubation with the presently disclosed peptides indicates the high potential of the peptides as an immunotherapy agent. Therefore, in at least one embodiment, administration or delivery of the peptides into tumors will result in pyroptosis and strong immune response. The adaptive immune system will be trained against specific cancer related proteins and suppress further cancer formation. FIG. 6C shows that the mechanism of the [II] peptides is dose and time dependent. The peptide nanofibers interact with the cell membrane and result in cell membrane damage and pyroptotic morphology (FIG. 6D). Propidium iodide uptake indicated membrane damage (FIG. 6D). Additionally, caspase-3 cleavage also indicates pyroptosis (FIG. 6E).

### Example 3

As mentioned above, the [II] peptide pair causes initiation of pyroptosis in a very short time on cancer cells. The immunogenicity of pyroptosis is a result of cell membrane damage and release of damage associated molecular patterns (DAMPs) from the cells, which creates a local inflammation and attracts the immune system. The difference of DAMPs initiated inflammation is, because there is no pathogen involved, it is known as sterile inflammation.

Supernatant of the OVCAR-8 cells was incubated with the [II] peptides and observed released Hsp90 and ATP, which is another DAMP. ATP is released from both apoptotic and necrotic cells, however, given that ATP is being used during apoptosis, released ATP from necrosis is nearly twice when compared to apoptosis. When released, ATP induces DC to produce IL-1beta and stimulates anti-cancer immunity. When cells were cultured with [II] peptides, the result was an increase in ATP secretion to extracellular matrix. As a result, the [II] peptides has shown the release of DAMPs that can create cellular and humoral immune responses. As noted above, the presently disclosed anionic/cation peptide sets can be used as adjuvants for variety of vaccines when they are simply mixed with them. Moreover, the peptides can be functionalized individually. Functionalization of the peptides with the whole Ovalbumin protein was shown, as explained above. Ovalbumin was observed on the surface of the OVA-peptide conjugated [II] nanofibers by using citrate coated AuNPs. Gold has more affinity to the thiol groups than citrate. The only thiol group in this system is coming from the cysteine of Ovalbumin protein. Citrate coated AuNPs will bind to the surfaces of the nanofibers if there is OVA displayed (FIGS. 7A-B). When there is no OVA, because there is no thiol group, AuNPs do not bind to the surfaces of the peptides.

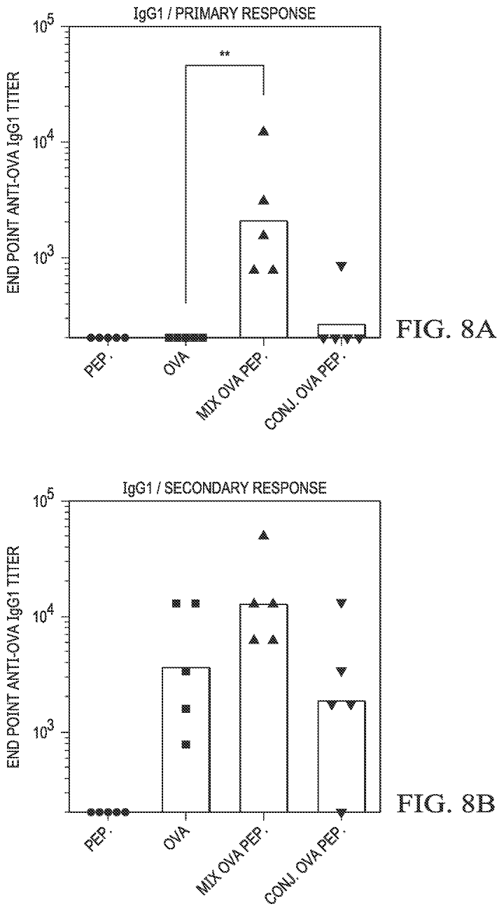

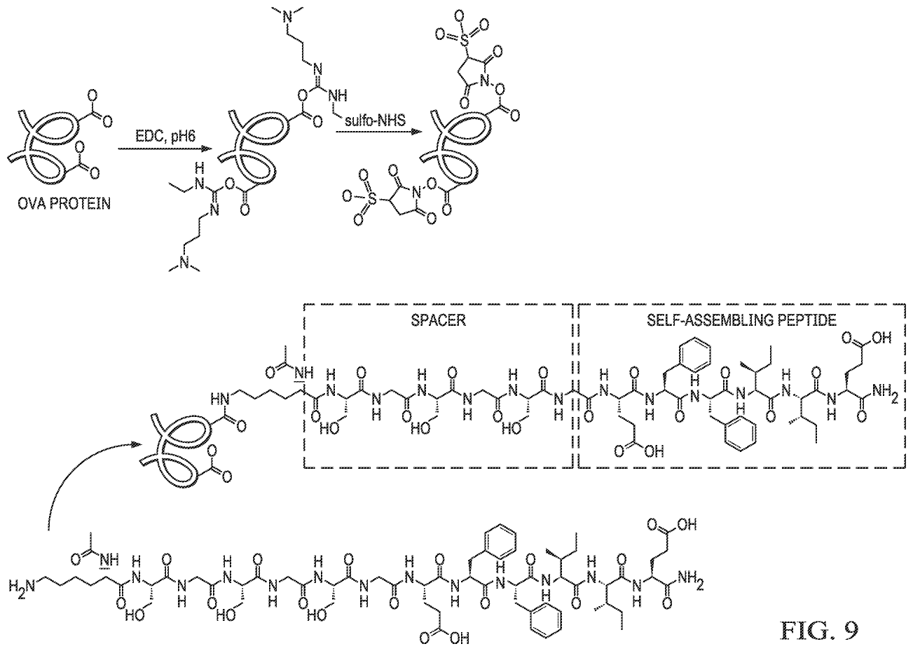

Nanofiber self-assembled from [II] anionic/cationic peptides which are conjugated to OVA ([II] conjugate), [II] anionic/cationic nanofibers mixed with OVA ([II] mixture), and [II] anionic/cationic nanofibers alone (Pep) were administered to mice. We observed antibody production against OVA on mice vaccinated with the [II] mixture (FIG. 8A). After the second vaccination, a higher amount of antibody production was observed, not only in [II] mixture, but also in the [II] conjugate treatment (FIG. 8B). A representative synthetic scheme for the conjugation of OVA to the [II] peptides via a spacer (linker) peptide having sequence KSGSGSG (SEQ ID NO:317) is shown in FIG. 9.

The nanofibers can be mixed with antigens of any pathogen (protein, inactivated virus, inactivated bacteria, etc.) and can hold it where it is injected together. Upon pyroptosis on the cells where it is administered, recruited immune cells will uptake the debris, including the antigens, efficiently, for humoral and cellular immune response. The nanofibers and antigens can be covalently conjugated.

In summary, in at least one non-limiting embodiment, the present disclosure is directed to a peptide composition which comprises anionic (negatively-charged) peptides and cationic (positively-charged) peptides, wherein the anionic peptides comprise Formula 1 or Formula 3, and the cationic peptides comprise Formula 2 or Formula 4. In this embodiment Formula 1 is X_{Ap}X₁X₁ₙX₂X₂ₘX_{Aq} (SEQ ID NO:1), Formula 2 is X_{Cp}X₁X₁ₙX₂X₂ₘX_{Cq} (SEQ ID NO:2), Formula 3 is X_{Ap}X₂X₂ₘX₁X₁ₙX_{Aq} (SEQ ID NO:3), and Formula 4 is X_{Cp}X₂X₂ₘX₁X₁ₙX_{Cq} (SEQ ID NO:4), wherein X_{A} is an anionic amino acid, X_{C} is a cationic amino acid, X₁ is a phenylalanine or tryptophan, or an analog or derivative of phenylalanine or tryptophan having pi-pi stacking properties, X₂ is a natural or non-natural amino acid, p=0-10, q=0-10, n=1-10, and m=1-10, wherein p+q equals at least 1, and wherein the anionic and cationic peptides having Formula 1 and Formula 2 are able to self-assemble, and the anionic and cationic peptides having Formula 3 and Formula 4 are able to self-assemble, when the peptide composition is subjected to a self-assembly-stimulating condition. In this peptide composition, X_{A} may be selected from L- or D-aspartic acid and L- or D-glutamic acid, X_{C} may be selected from L- or D-lysine, L- or D-arginine, and L- or D-histidine, X₁ is selected from L- or D-phenylalanine or L- or D-tryptophan, and X₂ may be selected from glycine, L- or D-alanine, L- or D-leucine, L- or D-isoleucine, L- or D-valine, L- or D-serine, L- or D-threonine, L- or D-tyrosine, L- or D-phenylalanine, L- or D-tryptophan, L- or D-methionine, L- or D-cysteine, L- or D-asparagine, L- or D-glutamine, and L- or D-proline. In this peptide composition, X_{A} and/or X_{C} may be a non-natural amino acid. In this peptide composition, at least one of X_{A}, X_{C}, X₁, and X₂ may be a D-amino acid. In this peptide composition, at least one of the N-terminus and C-terminus of the anionic peptide and/or the cationic peptide may be linked to a cargo molecule. In this peptide composition, each N-terminal X_{A} and X_{C} and each C-terminal X_{A} and X_{C} may be covalently linked to a capping group. In this peptide composition, the capping group linked to each N-terminal X_{A} and X_{C} may be an acetyl and the capping group linked to each C-terminal X_{A} and X_{C} may be an amide. In this peptide composition, each anionic peptide and cationic peptide may comprise a length in a range of 5 to 42 amino acids. In this peptide composition, the self-assembly-stimulating condition may comprise a pH ranging from about 6.5 to about 8.5. In this peptide composition, X₂ may be a hydrophobic amino acid. In this peptide composition, the hydrophobic amino acid may be selected from glycine, or L- or D-alanine, L- or D-leucine, L- or D-isoleucine, L- or D-valine, L- or D-phenylalanine, L- or D-tryptophan, L- or D-methionine, or L- or D-proline. In at least one non-limiting embodiment, the present disclosure is directed to a peptide nanofiber produced by exposing the peptide composition to a self-assembly-stimulating condition (e.g., as schematically represented, for example, in FIGS. 1-4). In at least one non-limiting embodiment, the present disclosure is directed to a hydrogel comprising this peptide nanofiber. The peptide nanofiber may be functionalized. In at least one non-limiting embodiment, the present disclosure is directed to a vaccine comprising this peptide nanofiber, wherein the peptide nanofiber comprises antigenic moieties for stimulating an immune response. In at least one non-limiting embodiment, the present disclosure is directed to a vaccine adjuvant comprising this peptide nanofiber.

In at least one non-limiting embodiment, the present disclosure is directed to a peptide composition which comprises anionic (negatively-charged) peptides and cationic (positively-charged) peptides, wherein the anionic peptides comprise Formula 5 or Formula 7, and the cationic peptides comprise Formula 6 or Formula 8. In this embodiment Formula 5 is X_{Ap}X₁X₂(X₁X₂)ₙX_{Aq} (SEQ ID NO:5), Formula 6 is X_{Cp}X₁X₂(X₁X₂)ₙX_{Cq} (SEQ ID NO:6), Formula 7 is X_{Ap}X₂X₁(X₂X₁)ₙX_{Aq} (SEQ ID NO:7), and Formula 8 is X_{Cp}X₂X₁(X₂X₁)ₙX_{Cq} (SEQ ID NO:8), wherein X_{A} is an anionic amino acid, X_{C} is a cationic amino acid, X₁ is a phenylalanine or tryptophan, or an analog or derivative of phenylalanine or tryptophan having pi-pi stacking properties, X₂ is a natural or non-natural amino acid, p=0-10, q=0-10, and n=1-10, wherein p+q equals at least 1, and wherein the anionic and cationic peptides having Formula 5 and Formula 6 are able to self-assemble, and the anionic and cationic peptides having Formula 7 and Formula 8 are able to self-assemble, when the peptide composition is subjected to a self-assembly-stimulating condition. In this peptide composition, X_{A} may be selected from L- or D-aspartic acid and L- or D-glutamic acid, X_{C} may be selected from L- or D-lysine, L- or D-arginine, and L- or D-histidine, X₁ is selected from L- or D-phenylalanine or L- or D-tryptophan, and X₂ may be selected from glycine, L- or D-alanine, L- or D-leucine, L- or D-isoleucine, L- or D-valine, L- or D-serine, L- or D-threonine, L- or D-tyrosine, L- or D-phenylalanine, L- or D-tryptophan, L- or D-methionine, L- or D-cysteine, L- or D-asparagine, L- or D-glutamine, and L- or D-proline. In this peptide composition, X_{A} and/or X_{C} may be a non-natural amino acid. In this peptide composition, at least one of X_{A}, X_{C}, X₁, and X₂ may be a D-amino acid. In this peptide composition, at least one of the N-terminus and C-terminus of the anionic peptide and/or the cationic peptide may be linked to a cargo molecule. In this peptide composition, each N-terminal X_{A} and X_{C} and each C-terminal X_{A} and X_{C} may be covalently linked to a capping group. In this peptide composition, the capping group linked to each N-terminal X_{A} and X_{C} may be an acetyl and the capping group linked to each C-terminal X_{A} and X_{C} may be an amide. In this peptide composition, each anionic peptide and cationic peptide may comprise a length in a range of 5 to 42 amino acids. In this peptide composition, the self-assembly-stimulating condition may comprise a pH ranging from about 6.5 to about 8.5. In this peptide composition, X₂ may be a hydrophobic amino acid. In this peptide composition, the hydrophobic amino acid may be selected from glycine, or L- or D-alanine, L- or D-leucine, L- or D-isoleucine, L- or D-valine, L- or D-phenylalanine, L- or D-tryptophan, L- or D-methionine, or L- or D-proline. In at least one non-limiting embodiment, the present disclosure is directed to a peptide nanofiber produced by exposing the peptide composition to a self-assembly-stimulating condition (e.g., as schematically represented, for example, in FIGS. 1-4). In at least one non-limiting embodiment, the present disclosure is directed to a hydrogel comprising this peptide nanofiber. The peptide nanofiber may be functionalized. In at least one non-limiting embodiment, the present disclosure is directed to a vaccine comprising this peptide nanofiber, wherein the peptide nanofiber comprises antigenic moieties for stimulating an immune response. In at least one non-limiting embodiment, the present disclosure is directed to a vaccine adjuvant comprising this peptide nanofiber.

In other non-limiting embodiments the present disclosure is directed to methods of making the peptide nanofibers described herein by exposing the peptide compositions to self-assembly stimulating conditions.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure. Changes may be made in the formulation of the various compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims. Applicants reserve the right to amend, add to, or replace the claims indicated herein below in subsequent patent applications.

1. A peptide composition, comprising anionic (negatively-charged) peptides and cationic (positively-charged) peptides, wherein the anionic peptides comprise Formula 1 or Formula 3, and the cationic peptides comprise Formula 2 or Formula 4, wherein
Formula 1 is X_{Ap}X₁X₁ₙX₂X₂ₘX_{Aq}(SEQ ID NO:1),
Formula 2 is X_{Cp}X₁X₁ₙX₂X₂ₘX_{Cq}(SEQ ID NO:2),
Formula 3 is X_{Ap}X₂X₂ₘX₁X₁ₙX_{Aq}(SEQ ID NO:3), and
Formula 4 is X_{Cp}X₂X₂ₘX₁X₁ₙX_{Cq}(SEQ ID NO:4),
wherein
X_{A} is an anionic amino acid,
X_{C} is a cationic amino acid,
X₁ is a phenylalanine or tryptophan, or an analog or derivative of phenylalanine or tryptophan having pi-pi stacking properties,
X₂ is a natural or non-natural amino acid,
p=0-10,
q=0-10,
n=1-10, and
m=1-10, and
with the proviso that at p+q equals at least 1, and wherein the anionic and cationic peptides having Formula 1 and Formula 2 are able to self-assemble, and the anionic and cationic peptides having Formula 3 and Formula 4 are able to self-assemble into a nanofiber when the peptide composition is subjected to a self-assembly-stimulating condition.

2. The peptide composition of claim 1, wherein X_{A} is selected from L- or D-aspartic acid and L- or D-glutamic acid, X_{C} is selected from L- or D-lysine, L- or D-arginine, and L- or D-histidine, X₁ is selected from L- or D-phenylalanine or L- or D-tryptophan, and X₂ is selected from glycine, L- or D-alanine, L- or D-leucine, L- or D-isoleucine, L- or D-valine, L- or D-serine, L- or D-threonine, L- or D-tyrosine, L- or D-phenylalanine, L- or D-tryptophan, L- or D-methionine, L- or D-cysteine, L- or D-asparagine, L- or D-glutamine, and L- or D-proline.

3. The peptide composition of claim 1, wherein X_{A} and/or X_{C} is a non-natural amino acid.

4. The peptide composition of claim 1, wherein at least one of X_{A}, X_{C}, X₁, and X₂ is a D-amino acid.

5. The peptide composition of claim 1, wherein at least one of the N-terminus and C-terminus of the anionic peptide and/or the cationic peptide is linked to a cargo molecule.

6. The peptide composition of claim 1, wherein each N-terminal X_{A} and X_{C} and each C-terminal X_{A} and X_{C} is covalently linked to a capping group.

7. The peptide composition of claim 6, wherein the capping group linked to each N-terminal X_{A} and X_{C} is an acetyl and the capping group linked to each C-terminal X_{A} and X_{C} is an amide.

8. The peptide composition of claim 1, wherein each anionic peptide and cationic peptide comprises a length in a range of 5 to 42 amino acids.

9. The peptide composition of claim 1, wherein the self-assembly-stimulating condition comprises a pH ranging from about 6.5 to about 8.5.

10. The peptide composition of claim 1, wherein X₂ is a hydrophobic amino acid.

11. The peptide composition of claim 11, wherein the hydrophobic amino acid is selected from glycine, or L- or D-alanine, L- or D-leucine, L- or D-isoleucine, L- or D-valine, L- or D-phenylalanine, L- or D-tryptophan, L- or D-methionine, or L- or D-proline.

12. A peptide nanofiber produced by exposing the peptide composition of claim 1 to a self-assembly-stimulating condition.

13. A hydrogel, comprising the peptide nanofiber of claim 12.

14. The hydrogel of claim 13, wherein the peptide nanofiber is functionalized.

15. A vaccine, comprising the peptide nanofiber of claim 12, wherein the peptide nanofiber comprises antigenic moieties for stimulating an immune response.

16. A vaccine adjuvant, comprising the peptide nanofiber of claim 12.

17. A peptide composition, comprising anionic (negatively-charged) peptides and cationic (positively-charged) peptides, wherein the anionic peptides comprise Formula 5 or Formula 7, and the cationic peptides comprise Formula 6 or Formula 8, wherein
Formula 5 is X_{Ap}X₁X₂(X₁X₂)ₙX_{Aq}(SEQ ID NO:5),
Formula 6 is X_{Cp}X₁X₂(X₁X₂)ₙX_{Cq}(SEQ ID NO:6),
Formula 7 is X_{Ap}X₂X₁(X₂X₁)ₙX_{Aq}(SEQ ID NO:7), and
Formula 8 is X_{Cp}X₂X₁(X₂X₁)ₙX_{Cq}(SEQ ID NO:8),
wherein
X_{A} is an anionic amino acid,
X_{C} is a cationic amino acid,
X₁ is a phenylalanine or tryptophan, or an analog or derivative of phenylalanine or tryptophan having pi-pi stacking properties,
X₂ is a natural or non-natural amino acid,
p=0-10,
q=0-10, and
n=1-10, and
with the proviso that at p+q equals at least 1, and wherein the anionic and cationic peptides having
Formula 5 and Formula 6 are able to self-assemble, and the anionic and cationic peptides having
Formula 7 and Formula 8 are able to self-assemble into a nanofiber when the peptide composition is subjected to a self-assembly-stimulating condition.

18. The peptide composition of claim 17, wherein X_{A} is selected from L- or D-aspartic acid and L- or D-glutamic acid, X_{c} is selected from L- or D-lysine, L- or D-arginine, and L- or D-histidine, X₁ is selected from L- or D-phenylalanine or L- or D-tryptophan, and X₂ is selected from glycine, L- or D-alanine, L- or D-leucine, L- or D-isoleucine, L- or D-valine, L- or D-serine, L- or D-threonine, L- or D-tyrosine, L- or D-phenylalanine, L- or D-tryptophan, L- or D-methionine, L- or D-cysteine, L- or D-asparagine, L- or D-glutamine, and L- or D-proline.

19. The peptide composition of claim 17, wherein X_{A} and/or X_{C} is a non-natural amino acid.

20. The peptide composition of claim 17, wherein at least one of X_{A}, X_{C}, X₁, and X₂ is a D-amino acid.

21-32. (canceled)
